SEQUENCE LISTING (78 sequences)

SEQ ID NO: 1 - DNA, 21 bp, Homo sapiens
aatttatcgaccacctgcaag

SEQ ID NO: 2 - DNA, 21 bp, Mus musculus
tactggcctgcctgtaaaaat

SEQ ID NO: 3 - DNA, 21 bp, Mus musculus
ggcctgcctgtaaaaatgggg

SEQ ID NO: 4 - DNA, 21 bp, Mus musculus
gccaccaagacacgccttgaa

SEQ ID NO: 5 - DNA, 20 bp, Mus musculus
attagacacttcagatagat

SEQ ID NO: 6 - RNA, 25 nt, Artificial Sequence
  Synthetic construct
ugaguguggcacagaaaccagaaaa

SEQ ID NO: 7 - RNA, 27 nt, Artificial Sequence
  Synthetic construct
uuuucugguuucugugccacacucagu

SEQ ID NO: 8 - RNA, 25 nt, Artificial Sequence
  Synthetic construct
gaguguggcacagaaaccagaaaaa

SEQ ID NO: 9 - RNA, 27 nt, Artificial Sequence
  Synthetic construct
uuuuucugguuucugugccacacucag

SEQ ID NO: 10 - RNA, 25 nt, Artificial Sequence
  Synthetic construct
aguguggcacagaaaccagaaaaac

SEQ ID NO: 11 - RNA, 27 nt, Artificial Sequence
  Synthetic construct
guuuuucugguuucugugccacacuca

SEQ ID NO: 12 - RNA, 25 nt, Artificial Sequence
  Synthetic construct
guguggcacagaaaccagaaaaacu

SEQ ID NO: 13 - RNA, 27 nt, Artificial Sequence
  Synthetic construct
aguuuuucugguuucugugccacacuc

SEQ ID NO: 14 - RNA, 25 nt, Artificial Sequence
  Synthetic construct
uguggcacagaaaccagaaaaacuu

SEQ ID NO: 15 - RNA, 27 nt, Artificial Sequence
  Synthetic construct
aaguuuuucugguuucugugccacacu

SEQ ID NO: 16 - RNA, 25 nt, Artificial Sequence
  Synthetic construct
uggagcgcugcaaguacuggccugc

SEQ ID NO: 17 - RNA, 27 nt, Artificial Sequence
  Synthetic construct
gcaggccaguacuugcagcgcuccaaa

SEQ ID NO: 18 - RNA, 25 nt, Artificial Sequence
  Synthetic construct
cagcucugccguuacuucccugcuu

SEQ ID NO: 19 - RNA, 27 nt, Artificial Sequence
  Synthetic construct
aagcagggaaguaacggcagagcugac

SEQ ID NO: 20 - RNA, 25 nt, Artificial Sequence
  Synthetic construct
agcucugccguuacuucccugcuug

SEQ ID NO: 21 - RNA, 27 nt, Artificial Sequence
  Synthetic construct
caagcagggaaguaacggcagagcuga

SEQ ID NO: 22 - RNA, 25 nt, Artificial Sequence
  Synthetic construct
gcucugccguuacuucccugcuugu

SEQ ID NO: 23 - RNA, 27 nt, Artificial Sequence
  Synthetic construct
acaagcagggaaguaacggcagagcug

SEQ ID NO: 24 - RNA, 25 nt, Artificial Sequence
  Synthetic construct
cucugccguuacuucccugcuugua

SEQ ID NO: 25 - RNA, 27 nt, Artificial Sequence
  Synthetic construct
uacaagcagggaaguaacggcagagcu

SEQ ID NO: 26 - RNA, 25 nt, Artificial Sequence
  Synthetic construct
ucugccguuacuucccugcuuguaa

SEQ ID NO: 27 - RNA, 27 nt, Artificial Sequence
  Synthetic construct
uuacaagcagggaaguaacggcagagc

SEQ ID NO: 28 - RNA, 25 nt, Artificial Sequence
  Synthetic construct
cugccguuacuucccugcuuguaag

SEQ ID NO: 29 - RNA, 27 nt, Artificial Sequence
  Synthetic construct
cuuacaagcagggaaguaacggcagag

SEQ ID NO: 30 - RNA, 25 nt, Artificial Sequence
  Synthetic construct
ugccguuacuucccugcuuguaaga

SEQ ID NO: 31 - RNA, 27 nt, Artificial Sequence
  Synthetic construct
ucuuacaagcagggaaguaacggcaga

SEQ ID NO: 32 - RNA, 25 nt, Artificial Sequence
  Synthetic construct
gccguuacuucccugcuuguaagaa

SEQ ID NO: 33 - RNA, 27 nt, Artificial Sequence
  Synthetic construct
uucuuacaagcagggaaguaacggcag

SEQ ID NO: 34 - RNA, 28 nt, Artificial Sequence
  Synthetic construct
gguagcuuuucuaacgcugagaugagug

SEQ ID NO: 35 - RNA, 28 nt, Artificial Sequence
  Synthetic construct
cacucaucucagcguuagaaaagcuacc

SEQ ID NO: 36 - RNA, 28 nt, Artificial Sequence
  Synthetic construct
gugaacugaguguggcacagaaaccaga

SEQ ID NO: 37 - RNA, 28 nt, Artificial Sequence
  Synthetic construct
ucugguuucugugccacacucaguucac

SEQ ID NO: 38 - RNA, 28 nt, Artificial Sequence
  Synthetic construct
cagaaaaacuuuuggagcgcugcaagua

SEQ ID NO: 39 - RNA, 28 nt, Artificial Sequence
  Synthetic construct
uacuugcagcgcuccaaaaguuuuucug

SEQ ID NO: 40 - RNA, 28 nt, Artificial Sequence
  Synthetic construct
aguacuggccugcuuguaaaaaugggga

SEQ ID NO: 41 - RNA, 28 nt, Artificial Sequence
  Synthetic construct
uccccauuuuuacaagcaggccaguacu

SEQ ID NO: 42 - RNA, 28 nt, Artificial Sequence
  Synthetic construct
ggaugagugugccuaccaucaccccauc

SEQ ID NO: 43 - RNA, 28 nt, Artificial Sequence
  Synthetic construct
gauggggugaugguaggcacacucaucc

SEQ ID NO: 44 - RNA, 28 nt, Artificial Sequence
  Synthetic construct
ccaucucacccugcaaagccuuccccaa

SEQ ID NO: 45 - RNA, 28 nt, Artificial Sequence
  Synthetic construct
uuggggaaggcuuugcagggugagaugg

SEQ ID NO: 46 - RNA, 28 nt, Artificial Sequence
  Synthetic construct
ccaauuguaaauuugcugaaaaauguuu

SEQ ID NO: 47 - RNA, 28 nt, Artificial Sequence
  Synthetic construct
aaacauuuuucagcaaauuuacaauugg

SEQ ID NO: 48 - RNA, 28 nt, Artificial Sequence
  Synthetic construct
guuuguuuguucacccaaauuguaaaua

SEQ ID NO: 49 - RNA, 28 nt, Artificial Sequence
  Synthetic construct
uauuuacaauuugggugaacaaacaaac

SEQ ID NO: 50 - RNA, 28 nt, Artificial Sequence
  Synthetic construct
aauaugaugcaaaguguacuaaaccaga

SEQ ID NO: 51 - RNA, 28 nt, Artificial Sequence
  Synthetic construct
ucugguuuaguacacuuugcaucauauu

SEQ ID NO: 52 - RNA, 28 nt, Artificial Sequence
  Synthetic construct
ccagauugucccuucacucaugugagua

SEQ ID NO: 53 - RNA, 28 nt, Artificial Sequence
  Synthetic construct
uacucacaugagugaagggacaaucugg

SEQ ID NO: 54 - RNA, 28 nt, Artificial Sequence
  Synthetic construct
aguagaagaauuccaguacugucuccaa

SEQ ID NO: 55 - RNA, 28 nt, Artificial Sequence
  Synthetic construct
uuggagacaguacuggaauucuucuacu

SEQ ID NO: 56 - RNA, 28 nt, Artificial Sequence
  Synthetic construct
ccaaaaccaguugcaccaccagcaccac

SEQ ID NO: 57 - RNA, 28 nt, Artificial Sequence
  Synthetic construct
guggugcugguggugcaacugguuuugg

SEQ ID NO: 58 - RNA, 28 nt, Artificial Sequence
  Synthetic construct
accaccuuccaguagucagcucugccgu

SEQ ID NO: 59 - RNA, 28 nt, Artificial Sequence
  Synthetic construct
acggcagagcugacuacuggaagguggu

SEQ ID NO: 60 - RNA, 29 nt, Artificial Sequence
  Synthetic construct
ccguuacuucccugcuuguaagaagaugg

SEQ ID NO: 61 - RNA, 29 nt, Artificial Sequence
  Synthetic construct
ccaucuucuuacaagcagggaaguaacgg

SEQ ID NO: 62 - RNA, 28 nt, Artificial Sequence
  Synthetic construct
auggaaugucccuucuaucauccaaaac

SEQ ID NO: 63 - RNA, 28 nt, Artificial Sequence
  Synthetic construct
guuuuggaugauagaagggacauuccau

SEQ ID NO: 64 - RNA, 28 nt, Artificial Sequence
  Synthetic construct
aaacauuguagguuuaacacucaaugua

SEQ ID NO: 65 - RNA, 28 nt, Artificial Sequence
  Synthetic construct
uacauugaguguuaaaccuacaauguuu

SEQ ID NO: 66 - RNA, 28 nt, Artificial Sequence
  Synthetic construct
auguacaagaccggacugcacauucuac

SEQ ID NO: 67 - RNA, 28 nt, Artificial Sequence
  Synthetic construct
guagaaugugcaguccggucuuguacau

SEQ ID NO: 68 - RNA, 28 nt, Artificial Sequence
  Synthetic construct
cuaccaucccaccauuaaugucccacca

SEQ ID NO: 69 - RNA, 28 nt, Artificial Sequence
  Synthetic construct
uggugggacauuaauggugggaugguag

SEQ ID NO: 70 - RNA, 28 nt, Artificial Sequence
  Synthetic construct
accacgacaugccuugaaauggauucga

SEQ ID NO: 71 - RNA, 28 nt, Artificial Sequence
  Synthetic construct
ucgaauccauuucaaggcaugucguggu

SEQ ID NO: 72 - RNA, 28 nt, Artificial Sequence
  Synthetic construct
auggauucgaccucaaaccagcgaauaa

SEQ ID NO: 73 - RNA, 28 nt, Artificial Sequence
  Synthetic construct
uuauucgcugguuugaggucgaauccau

SEQ ID NO: 74 - DNA, 477 bp, Homo sapiens
ggtagcttttctaacgctgagatgagtgaactgagtgtggcacagaaaccagaaaaacttttggagcgctgcaagtactggcctgcttgtaaaaatggggatgagtgtgcctaccatcaccccatctcaccctgcaaagccttccccaattgtaaatttgctgaaaaatgtttgtttgttcacccaaattgtaaatatgatgcaaagtgtactaaaccagattgtcccttcactcatgtgagtagaagaattccagtactgtctccaaaaccagttgcaccaccagcaccaccttccagtagtcagctctgccgttacttccctgcttgtaagaagatggaatgtcccttctatcatccaaaacattgtaggtttaacactcaatgtacaagaccggactgcacattctaccatcccaccattaatgtcccaccacgacatgccttgaaatggattcgacctcaaaccagcgaataa

SEQ ID NO: 75 - DNA, 3556 bp, Mus musculus
ggggacgcgcacggcggaggcggagcggcggcggcagcggcggcagcggcagcggcagcggcgtagggggcccaggctgcagggtggcagcccgcggcgggctccaggtaaccgaggcgccgcgcagtgccgagccggccgcccgccgccgagccatggaaatcggcaccgagatcagccgcaagatccggagtgccattaaggggaaattacaagaattaggagcttacgtagatgaagaacttcctgattacattatggtgatggtggccaacaagaaaagtcaggaccaaatgacagaggacctgtccctgtttctagggaacaacacaattcgattcaccgtatggctccatggtgtattagataaactgcgctctgtcacgactgagccctctagtctaaagtctcctgacgccagcatcttcgatagtcacgtgccttcaaacaagagcagtttcagtcggggagatgagagaaggcacgaagctgccgtccctccccttgctgtttctagttctagacctgaaaagagggattccagagtttctacaagttcacaggagcagaaatccactaatgtcagacattcatatgatgatggagcttccacccggctaatgtcaacagtgaaacctctgagggaaccagcaccctctgaagatgtgattgatatcaagccagaaccagatgatctcattgatgaagacctcaattttgtgcaggagaatcccttatctcagaaaaaacctacagtgacacttacatacggttcttctcgcccttctattgaaatttatcgaccacctgcaagtagaaatgcagacactggtactcacttaaacaggctgcaacttcatccgcagcaaagcagtgctcacgctgccaagcagctggatgtacaaagcagccaggtatccgaagcaggacggttgtgtgagccaccagtgcttagcagcgtagaagacacttatagccccttcttcagaaacaacttggataaaatgagtattgaggacgaaaactttcgaaagagaaaattgcctgtggtaagttcggttgttaaagtaaaaagatttagccatgatggagaagaggaggaagaagatgaggattatgggacccgcataggaagcttgtccagcagcgtgtcagtaccagcaaagcctgagaggagaccttctcttccaccttctaaacaagctaacaagaatctaattttgaaggctatctctgaagctcaagagtctgtaacaaagacaactaactattctgcagttccacagaaacagacacttccagttgctcccagaactcgaacttctcaagaagaattgctagcagaaatggtccaggggcaaaacagggcccccagaataagtccccctgttaaagaagaggaagcaaaaggagataatacaggaaaaagtcaaggaactcaacagaggcaattgttatcccgactgcaaattgatccagtaatggtagaaacaatggagatgagtcaagattactatgacatggaatccatggtccatgcagacacaagatcatttattctgaagaagccaaagctgtctgaggaaatagtagtgacacccaaccaggattcggggatgaagactgcagatgcccttcgggtcctttcaggacaccttatgcagacacgagatcttgtacaaccagataaacctgcaagtcccaagtttatagtgacgctggatggtgtccccagccccccaggatacatgtcagatcaagaggaggagatgtgctttgaaggaatgaaacccgtaaaccaaacttcagcctcaaacaagggactcagaggtctcctccacccacagcagttgcatttgctgagcaggcagcttgaggacccagatggtagcttttccaacgccgagatgactgacctgagtgtggcacagaaaccagaaaaacttctggagcgctgcaagtactggcctgcctgtaaaaatggggatgagtgtgtataccatcatcccatttcaccttgcaaagcctttcccaactgtaaatttgctgagaaatgtttgtttgtgcatccaaattgtaaatatgacacaaagtgtactaaagcagattgtcccttcactcacatgagtagaagagcctcgatactgactccaaaaccagtgtcgtcaccagcaccgtcttctaatggccagctctgccgttacttccctgcttgtaagaaaatggaatgtcccttctaccacccaaaacactgtaggtttaacactcagtgtacgagacctgactgcacattttatcaccccaccattactgtgccaccaagacacgccttgaaatggattcgacctcagagcagtgagtgatgccctagtcctacctggcagaagatcatgcagtttgaaagcttccatcttctgatgagagatgttctacagaacttgtcacgtctttgaaatttagaatatattgctttcataatacgaattttactgccccactgaagtgtctaatttttcaagtttgtaagtttattaagtggtttcaacattttttgtttgttcgttttgactatgaaaaagacagtttaaagaaaagccaaattctattaaaacatttgcggcatgtttgtacattgctgtttaatatcatttttggtaatggtacttgcagcttagggctgtagtgctgtgggaaggccagtgtcctcagagctgaagcacttttcagcttttcccaaaggtaatgcagtgtctgtaacccagcgtggtaacagtggccaggctttgaaactgaggcagctttggaacaactagtttaaatttctttttttagtgtctaaatgaatttgctctgagaagcataatgcagactttattttgagtgctactttggtagagtggaccgaggtcctgtgcctttctgaaagtgagcagagacatggtcataaagggtaagcatagttggaatgacgatgtaaaaatatatggacagttctttggaatgctcccatttactattagcttatcattttataagtaattttggagggactacattatcacaaaagtatacaaaaatttttacaggcatatgtacagaaagtatcagaaaacagactttgaactcacaagaatataaatatacgtatatattcccatattctgaaaaatatcatcagaaataaccccacagaaaatatacttatgttattactaaagatcattcttgaaatgtagaagttgagatttaagtggtatattttaaatgacagaactatattgcagagataggaaggtaaacttgacaataggatgaaacttggcctactgtactatggagttttatgtgtggtttttgaaactgttaaggcaagatgtgtcatgttttagaactaaataacagacaactgatttcaaaaacgtgttgttttaaaaattaaagtgtaaacggtggttagcaaaggggataataaaagctcaaacattttgaggaccaaatttaactgttaagatacaataaagtcacatctataaaagtctgtgtttaataatgtgaa

SEQ ID NO: 76 - DNA, 18153 bp, Homo sapiens
ggaggcggtggtgtcccggctgcggggtaggagtccgcggcagcctccgggtaagccaagcgccgcgcagtgctgagttcccgcacgccgcagagccatggagatcggcaccgagatcagccgcaagatccggagtgccattaaggggaaattacaagaattaggagcttatgttgatgaagaacttcctgattacattatggtgatggtggccaacaagaaaagtcaggaccaaatgacagaggatctgtccctgtttctagggaacaacacaattcgattcaccgtatggcttcatggtgtattagataaacttcgctctgttacaactgaaccctctagtctgaagtcttctgataccaacatctttgatagtaacgtgccttcaaacaagagcaatttcagtcggggagatgagaggaggcatgaagctgcagtgccaccacttgccattcctagcgcgagacctgaaaaaagagattccagagtttctacaagttcgcaggagtcaaaaaccacaaatgtcagacagacttacgatgatggagctgcaacccgactaatgtcaacagtgaaacctttgagggagccagcaccctctgaagatgtgattgatattaagccagaaccagatgatctcattgacgaagacctcaactttgtgcaggagaatcccttatctcagaaaaaacctacagtgacacttacatatggttcttctcgcccttctattgaaatttatcgaccacctgcaagtagaaatgcagatagtggtgttcatttaaacaggttgcaatttcaacagcagcagaatagtattcatgctgccaagcagcttgatatgcagagtagttgggtatatgaaacaggacgtttgtgtgaaccagaggtgcttaacagcttagaagaaacgtatagtccgttctttagaaacaactcggagaaaatgagtatggaggatgaaaactttcggaagagaaagttgcctgtggtaagttcagttgttaaagtaaaaaaattcaatcatgatggagaagaggaggaagaagatgatgattacgggtctcgaacaggaagcatctccagcagtgtgtctgtgcctgcaaagcctgaaaggagaccttctcttccaccttctaaacaagctaacaagaatctgattttgaaggctatatctgaagctcaagaatccgtaacaaaaacaactaactactctacagttccacagaaacagacacttccagttgctcccagaactcgaacttctcaagaagaattgctagcagaagtggtccagggacaaagtaggacccccagaataagtccccccattaaagaagaggaaacaaaaggagattctgtagaaaaaaatcaaggaactcaacagaggcaattattatcccgactgcaaatcgacccagtaatggcagaaactctgcagatgagtcaagattactatgacatggaatccatggtccatgcagacacaagatcatttattctgaagaagccaaagctgtctgaggaagtagtagtggcaccaaaccaagagtcggggatgaagactgcagattcccttcgggtactttcaggacaccttatgcagacacgagatcttgtacaaccagataaacctgcaagtcccaagtttatagtgacgctggatggtgtccccagccccccaggatacatgtcagatcaagaggaggacatgtgctttgaaggaatgaaacccgtaaaccaaactgcagcctcaaacaagggactcagaggtctcctccacccacagcagttgcacttgctgagcaggcagcttgaggacccaaatggtagcttttctaacgctgagatgagtgaactgagtgtggcacagaaaccagaaaaacttttggagcgctgcaagtactggcctgcttgtaaaaatggggatgagtgtgcctaccatcaccccatctcaccctgcaaagccttccccaattgtaaatttgctgaaaaatgtttgtttgttcacccaaattgtaaatatgatgcaaagtgtactaaaccagattgtcccttcactcatgtgagtagaagaattccagtactgtctccaaaaccagcagttgcaccaccagcaccaccttccagtagtcagctctgccgttacttccctgcttgtaagaagatggaatgtcccttctatcatccaaaacattgtaggtttaacactcaatgtacaagaccggactgcacattctaccatcccaccattaatgtcccaccacgacatgccttgaaatggattcgacctcaaaccagcgaatagcacccagtcctgcctggcagaagatcatgcagtttggaagttttcatgtactgatgaaagatactctacagaacttgtcaaatctttgaaacttggaatatattgctttcataatatgaagttttattgcctatctatctgaagtgtctaatttttcaagtttgtaagtttattatgtggttttaacattgggtgtttttgttttgtttttactatgaaaagacagcttaaggaagagctaaattctgttaaaatatttggggcatgtttgtgcactgctgttgtgaggatcagcatatgaaattgacatcatggttagtcatggtactgcagcttagggggctacacggttgctgtgtgagtggagagatgcagtgaggcagttgtcattattctaaaaattgtactactttcacttttcccaaagattatataatgttcataatccaccatgaaaacagcattggccaaaggtactgaggctgcttaaaatattcaattctgctttttaatttttaagtgaatttagtttgaaaagcatgattatacaggcctctcaggctgagtgctactttggtaaagttcccagttttcctgccttctgtgacaggatgaatgaggtgggtatggacagtggaggcagctggaatggcaagtgcagaaaataggaacagttctatacagtgctctcatttactaataacataatgccttctaaataatttttttgggaaactacattatcacaaaattatacaaatttttttacaagtatttacatactgtatctgaaaacagactttaaagtcacaagattataaatgtacatatatattctcacattctgaaaaataacattctcagaatccacagaaaatatacttagttactactgaagataatttttgaaatgtaaaaattagatttaaatagtatattttaaatgacagaactataattacagagatcagatcagataggtaaactgcaagatagataggatgaaacttttggcctactgtattacttacagagtttttttgtgtgtggtttttaaaactgttaaggcaagaagtgtcaaatgctttagagttaaataacagatcactgatttcaaagacttggtgtatagtgttaaaaattaaagcttaaaaggtggttagaaaagtggattaatgcaaaaggggtaataaagactgcaacattctcaggaccaaattaaactgctaaaaaaaaaaaaaaagttcattgacttgcttagtcgtatactcaaatgatgataaacctacatgtgcaaaggctcacgtttaagattgtcaagccagcagtctactgttgtgttgccattgcttttccattgggagaagaaagaattaaccagtcattaaaccatttggtaagttgcactttgctgtgctgatcccacaggaaaggcttgaaacacgagaagcagcaaagacagagcacacaagtgcataaggctgttgtcttcggcttgggtgaaatgacagttcctcttcattctaaaggtttactccattgaatttaaggcatttgttcattccagtgttgagatgctttgcatctctgcagaagaaatttattttaaattgtttaaatatctggaaatacttttagctatcatttataaagatagttttgttctcagtttcactataaattatagaacaaatgggaaacaagggtttaatttagttcagccattttacaaggaaataataaaatactaaaatctgattgttttttgctatttaatagccactgcccagacacatatttaagagtttaatctttcagttgctatggcttatgaacaagctaaggttgaccataaaacatttgttggatgacgtggtttaaaatgatcaccacaaaaagggaccacaaaaaaaggaaggaaatgagcatggttggcgattggaagcaagggtaccagagggcacagtgtgctttggcatgcattttatacataaaatgaatggaacaaaaggtgccagaagtcccaggttacacaatcaggagcttagatactgcacacaaaaataattatctgggttaaaaaagtaaacatagggcagattctatatggcctatcatgtttcttcaccttcccctcgttgctggctgatacagcgaggtggtcagctgatgactacttagtcaatatgacctttagtcgtgaaactgacagcagcagtgattaaggctgacttaatcaggttggccactttgaaggacagaaatgcagtggaaacagttttattctatgtagtttacatgcttaaggttacagagtttctacctgcactgtaatggaaatataatttctctgtagccaaaagctggcaaacttgacccagagggaaaatttaaaactgcagcaggctcaaatgtagagtatttttctttttatgggcaggttgttcagggatttttttcctcctttaatttattgactgactgtaaatacatgagtagaaacttaatagtcatgtatttcaaaatttggcttaatttaggagaatccactgatgaacaagtaccaacttacgtttcaagcttcttagccccataatcagtccttcagccacagctatttagagctttaaaactaccaggttcaatcactggttatgctttctgtgatgtaatttagtcatttctatttttagtattaaccaagtattagacacagaaaataggtattaagaatcttcatatatcctgtcagaccaaatgggattccaggaacctaaagcgatctattatgctataaagataattaacacattaaaaactcatagggtcaatacagcatcttaaacctcacacttagaaaaatatatttttaaatagcagtctacataattttcaatcttcaggaaactacagataggctagacagcgaattcctgaatgatgagtagtgatctttggcagcatttaaagtgaaaagaaataaggatctaagaattcagccctaatccactaaaaaaaggaattctaactgacaagtttttacaaatggagttgggctcattcattttggaaataaacctatggagtggcacacatctaaacaaattttcccaatagaaaaaaggctataaaaattttattccaagagtgattaaattgtataatgttgtatatgtgaatttaacacttttgtttacatgttaaacaaatgtgtatatattagactacattaaatatgcaattctttcttccagttaaatactgttgctccctaaaacccttacattgtacaccattgggaatgattgttcatcatactacttttccattagtgaggctacagttatgttttaaatgtgcgattacagagatggcatctgaacataaactgatggctcgaaaatgaaaatggaaatgtagcagccatatactgctaactttggatctgttcctgaattcaaaactactaggagaaaagtgtcctttataaaaaaggaccttattaatgcctaaaaaacatcatattctctaggaaagcttgtgtctgtttccttagggaaaatgtttgccttttaaaaactgtgatcctttaggatgatcatgactttccctttccttatggaaatgcaagaataaaatatttcattaaacaatgaaccttgaaaataaaatataaacattaagaaaccattttgctaaaaagataatgaaaattatccaaattgggtttttgagttcttctgtaaagagtgctctaccctaaattttcccagcaggtctgccgaaatcacacacttcccaatacagggggacttggcctttaccatcaagtattcgatccttccttgaaatggcattatctggcagtgtatggattacggattatacccagtgcatatagcaaatattttgaacagatcagtctttcactattttgatgattctgggcatttctccctgttacagtcttgggttagcaccacttgaccatgcagggttgggttttggtttttcttctctgtaattctggtctcaaagttaatttctgtagtcatctcagcatctctcagtgaggtgtatgtacacatttccagacaaataagctgcaatcagagaagaaaattgcagggagttaattatgtttttagattttcataacagtttaatatttttcagttgtgctttcaggttacatgtgtaatatttttcctctttaactccttttattctgtatttgcataaatatgagattctgaagagccatctggttatactaccttctactaatgttgactagctgatttcataaaccaaagctgtaggagttgttgtattaagtctcttaactagtaacatagtctgctcttcatgggctgagaaagttactaacctgcagtcatcacctccagcactaacaacatgtcgatcaccactggtaaatcgaatatttgtcacatggggcgaatgacccaagaaccttttgtgttttgcctaaaaaacaatgacagacaagctcagggcatttggtgcacacagaagtcaaaggctcttattaggaactataatctctatgacaagagctgtggagagagtagggagttagcaccgcagccagtgattagaatgcttttcagcatgagtagtggatctgcaaaaccaggctgtgtgggcagtcagatgtctccaggtactctgaccatttttctctaaggaaaagcatttgaaatttgataactgattataggtttggtgaaaagctaattacagcttttgtaggatggttccaaagatggtattactcgagggagaggatttgtttctaatagcttttatttcaaagtaaatagatttagaaagtttggggaaaaatttagaaattaggacaaaacattttaaatatatggggaaaagtgctgatgataagacatcaaaattaggagtaaactgataatagtaaacaaaacacaaacttacaaatttttctggacatgggaagtcaaataacttaaccatgccaaagtcatctcctgtaacaagactgattcctgaatgagatacacaggcacagttgacatcagctttctcagcatgtctggaccagattcccaaaacctcatctcctagaatactagagggaaggaacaaaagaaaactcatcatggcaagtgcgggcaggttgactatattcaaaaagtttcttggcaattaatctctaagtaccctatcatgttacttaaaatacaggaagtaaattatggtaagttgtttggagacctgaatttcatcaggatatcaactcctgccttttaaaaatgacattttataatttgaagggtttctagattaatctttttaagattaaagtagtactttatgaaaactgatagaactattttttctttttttttttttgagacggagttttcgctcttgttacccaggctggagtgcaatggcatgatctcggctcaccgcaacctctgcctcctgggttcaagcaattctcctgcctcagcctcccgagtagctgggatcacaggcatgcgctaacatgcccggcttattttgtatttttagtagagacagggtttctccatgttggtcaggctagtctcgaactcccgacctcaggtgatcacccccgctcggcctcccaaagtgctgggattacaggctgagccaccgcgcctgactgaaaactgatagaactatttttcaaattaaaagtgctacttggctgggtccagcagcacataccagtaatcccaacattttgggaggctgaggcaggaggactgcttgaggccaagagtttgagaccagcctgggcaatattgtgagatccctatctctacaaaaataaaaatgacttatgacataggaattaaaaaaatttcagagatggggtcttgctatgttgcccaggctggtatcaaaacttctaggctcaagtgatcctcccacctcggcctgctacatcagagattacaggcatgagccactatatgcctggctgatacaggaatttgatggcatttttcattggccaaaaaaatggatagtcatggttacctgtcatacagccaggaaatttgaacaaatttggaagctttgacttctaatagattcaagatagcattcctttagatagagaattaataacagttgcttaacagcacccaatacctttttgccagtcattaaatttagcattaagaaaaatatcagggtatctttaaagttaaaactttgatttccttaaaaaaaaaacttgataaatcatggaaactgataaaacatggaaatatattcaataaaaaggggtcccaacatgaacataccatttcaaaatatggtaacaaaaacttgaaactcaattactattccttattggaatggctctaacagttcagaaataggattttctaactggccttcaaagtcagttcttgccttgtgaatatataagtatttacctagtccatgtagcccaagtaattctgtcaatagcggcatgatccataagatgttttcctgaaggcacttcatagacatgccgtttatagcagccactagagacctttttcatcagattaaaatgggacaagaattccattaggtgagagacaaaatccacagggggtttacagaatactagcatattgctacttgatttacatgtctaacattattaagtatgcaaaagatcactacaaaaacttaataggagaaaagctctgataagtgggggaggaaaggggagctgtaggtcagaaggtacaaagggaggagttgagaagctggagctctggagctcaggaactttaaatgcattcactaacacgaaatgtaaaagcagaagaacttgccacctgggtatacagtattggtactgtacctggagataactgctatctgcagagaagtccatttgaatgacaaagcttggaatgtctttgcagtagctgattctgttaagagtggggcccagcgttaggtcataaaaatccactgagttctcactagaacctactgccagataccgggaatccggactaaatctgaatcaaaacaaaacgtaaaaagtattagaccacatgaagtattataaatacttaagatcagtgacttttcctttctagttcttaaaagtaacgtgtgataaggcctcaaatagatttacctgtcagacacaactgatcatgtatactgagattgtctgggttacatgaaataaggaagctttatattttacttaaattttaaatatttccccaattgtcatctcccaattcctttaaaaacgtctaatggcttaaaaaaactttcttaggccaggcccagtggctcacacctataatcccagaactttgggaagcggaggcgggcagatcacctgaggtcgagagtttgagaccagcctgaccaacatagagaaaccctgtctctactaaaaatacaaaattagccaggcatggtggtgcacgcctgtaatcccatctactcgggaggctgaagcaggagaatcgcttgaacccaggaggcacaggttgtggtgagctgagattgcaccattgcacttcagcatgggcaacaagagcaaaactccaactcaaaacaaaacaaaacaaaatttaattttttaaatagaggcggggtctcactatggtcccaaactcctggcctcaagcaatccttcccccttggcctcccaaggtactgggattacaggtgtgagccacaacacccagtcagaacatctcagcttttaaaagccattagcattacataattaataagctaacaattcattaagatagttttcttccatctggaaaaaacgttgtcttaatattaagcaaagaacacagcccagcttaactaacctccagttattaaggtgaaatgacacaacttgaatcttggaagaagaattttttttttttgagacgaagtctcgctcttgtctcccaggctggagtgcgatggcgcaacctccgcctcccgggttcaagcgattctcctgtttcagccccctgagtagctgggattacaggcgcctgccaccacgcccggctgatttttgtatttttagttgagatggggtttcactatgttggccaggctggtcgagtactcctgacttcaggtgatctgcctgcctcggcctcccaaagtgctgggattacaggcatgagccaccgcgcccggcctgaagaacttatttaaaagacaaagtgaaatgctatttgcctagcaatctttggagtcatatgggacaattcagtctcttgaaatggcccatgagtcttactgaggtacgatagagacatgtaaaagctaagggaagccactgttactattttatatattgaagttctgaggaaggtttcatttgtaaaaggattttactgatgaaaagtgtacaagcttttgacagacctagattcaataatcttatctactgatcacacggaagtactccgtaaatggtagccactgttgaaaaatgcttaagcactgaaaaacaaaggtttaagaaacatttaaattaatttggattctggaacatttaatcaataggtattgattaaattaatgaactacatattcccaaactgaggttactaagagaagatatgtttgaaatcacaactttagttttccagggtgacaacttttgaagggcagatagctctcttgtattacagtgggagatacctcttggtgggatgaacttaatggacatggctaagtgttaacatgaattcatcaaacattacctactagtacttgctattatagttggtgcccagtgggtttataatttagcaagaagaattaagtagtatacaaacagccatattttagcatacaatttataatacgggaaatgctacaggccctggggacctctttttgaaggcaaggctatggaaaattttacaaatggaagttaaatcaagtatatactagaaactctattccatttgttcactaacctgatatcatggattgcacatctcctgtctctcttctttccccatatttttagagaactcactagtaaaatgataaattctccatttttcattccaatagccaccatgtccccttcagggctgtaacacacagtacgagcagcatgtcccaaattcactttgtttaacatcttctgcatttaaaaaaaaaaaaaaaaagagtcataggaaacattaagtgaagtacttctaaattataccagtttcccctcaaaatgctcaacagaattctggcagttctttaagtactagcaatttagaacttccaacttttctttttagaagttgtaacctcttttaaaaaaattatctgtacttactttatcagcaatatcccaaagtctcactgtcccatcttctgcagcagaaaggaaaaaatccctggaaggatgtgttgctagtccccagattggcccatccacatgaccgttaactaaaatattacaagctgcatttttctctccaacttcgattatttcagcattccttgtcccaacaaggatcttgccctgaaacacaagcaggaccaatacagtgaatgtaatacaacagctgcttttcttcttcataatataaaaatgaccctattgacctgctttcagagaactttttgctttgagctaatctagtagcaaggcagtcattagctcatgcaaatttttctatgactacaggcacacatctatctgtaagcacaatgggctagattacatattagagtccatgctacagaatagaacttttctgtggcagtacacctggattcttcaataatcaaagtttttatttgataatcttaggatttccaaactggggtcagtgcagtgggatataggaaaaaataatagaatttattttttagttaaaaagtaaaagcttaactacaatttaatatgcaggctgaagataatatccgtatgatttataaatacacttaataagtacaaacacgctcaaaaattttcataggagttgtagttttgaatttttattttgaaattgacacataattatacatatctatagggcatagggtaatacgcataaccatcacctcagacatttatcatttctttgtgatggaaactttcaaaatcctctcttgtaaatacctgaaaatacataaatacgtgattcttaactatagtcatcctacagtactacagaatactaaaacatactattcctatctggctgtgtaaacttgtatcctttaaccagtccttccctatccccctccccctcccccttgtccgcctccagtaaccactattctactctccacctctgtgggatcaacttttttagtttctgcacaggagtgagaacatgtatttatctttctgtgcctggcttatttcacttcacatcatgtcctccagtctcatccatgttgccaccaagaatgacagaatttcattattttttatggctgagtagtatttcattgtttgtttactgcacgttttatctagggaatgtgtgtttttttaaaaaatggagacagctgtcctaatatgagtcaactgccaagggctttcaattatgtctactagagttgttaaattggcagattctagaaaatattggaggtttacatacagtatttagacagaatagcttcctagcttatgcaccacactggtgctaactttggcaaagaaagcagcaaagacagagtaatgttggcaagcaaatccatcgttatgcattattaagtattgttcattaggctgcaaagggtgagggaatcacagtaataaccactttctgttttctgctgcactgtatcagctcatggaacatcttactttgcctctgcacacagaacgaacacaatctgtggcttgtcctgtctcaagcctgaaggcacggcaccgcctcagttcctgatcccacagtttaaccgctcctccttcttttgacctaagtaaataaccaagccagagtaagtgttcattattggctactataatttttattataaacaaataccaagttataagcagaatcttttttttttaaaaaggccctgatatttataatttacctctaatattcttgtaaactttctatggcaatttgaggatatactatatctcagtcaaaataaacatccagtttcagtgaattttattttgagaaatactctttttttctgacatgagcataattttatttagcctctacaatacattacaatacattatcctctctcataatacttttttttttttttttaagatgtagtctcgctctgtctcccaggcttgagtgcagtggcatgatctaggcttattgcaacctctgcctcccaggttcaagcgattctcctacctcagcctcccgagtagctagcattacaggtgtgcaccaccacacccagctaatttctgtatttttagtagagatggggtttcaccatgttggccaggctggtctcaaataccttgacctcaggtgatctgcctgcctcggcctcccaaagtgctgggattccaggtatgagccactgtgcctggcctcataatacttcttgattaggaagatgtaaaaaaacaattttattaaaaggataatggaaatgtaaggcaaaataatagaattacaaatgctatgctacagagttgatttatttatttttttgagacagagtgtcgctctgtcacctggcctggagtgcagtggtgtgatctcggctcactgcaacctgtgcctcccaggttcaagcgattcttctccttcagcctcccaagtagctgggattacaggcaccatgcctggctaatttttgtatttttagtagagatggagtttcaccatattggccaggctgatcccaaactcctgacctcgtgatccgcccacctcggcctcccaaagtgttgggattacaggcgtgagccactgcaactggcccagagcttatttttgaaggccaaaacagaagcatatttattccctatcaggtgttaaaatatctcactggaacagtttagcaggcttctagtgagtgggggtgtgcaggagtaaatgacgtgggaaatacaagtgttggaggacgaaatagagcccatttatggattttattcctggaagggctgaaaaatgtattccttccttttctgctagatgaattgcttgtctgaaagcatgcctatgtgcattcttcctttatgtaaaaggcacaaattctgcgcttgtgtttaattaacatatgtgggttctttcaatcctgtattgaaatgtacttcttagtcaactatatgtcacatttttttttgtttttgtttttgttttttaaatggggtctcactctgtcacccaggctggagtgcagtggcaccatcacagctcactaaagccttgacctccccaggctcaagtgatcctcccacctcagcctcctgagtagcagggactacaggcatgtgccaccacacccggctaattgttgttttttatagcgatggggtttcaccatgttgcccaggctggtcttgaactcctgggctcaagcgatccacctgcctcagcctcccaaagtgataagattacaggtgtgagccactgtgcctggcctacatgtcatgtttcaacatgcatatgactatgttggtgacaaatcaaatcataagtatctggttactgttgggagatttgaaaatcactcagaagagacctcttctcaaattttgaggtcttgtataaaacagtttaaatttgcctcaagcaaaaggaaacaaggcagttctctctagttccctcatccttttctaaagcaacaatgtgcattctactccttagaatccattctgaacaaaaagagagcaggcagtcaaaatacaaccctggctccagattcccccatgggcctcctactcagcaaatcatacacaggcatacagacattaagaaaagtaactcaacttgtaggacaactacctatccacacctcagaaaaagtatcaccccaacatgaaaaaaattggaagtgaattaagaccagaaatgagaatcaaatagaaggcacataaaaggtaataaaggagaagcatatgaggaggaaggtcggagaggacactctgtgtagcctagaaacaactagaataattaactgcaaacctcaggtaggtcacaaatgcataaatattctgtgaaaagaaagaggactcacggcctttcctttcccccagtcacgataagtccatctcgcagggtggtgtacatggcaaacacaggcccgttgtgagctctcgccacgattctacacaatatgtgatctttccacacacagacatcaccactgatggtacctgtaaacgtcaagttattctgaaaaggagtgggggagggggagacaaactcatcaaaagttcaaatagagtttaaatagataattttctatgtatgtgtaatgctgtctcacccttgatacaaagagcatgcatcgtgtagtggcagcagcactgaattcacgagtcaggaaacctgaacgggaggcttagctttgtcaggaccttttcctttccaagtctgttgcttattagctagaataaccttagacaattcttcccttccaattctaacatactataattctagggtttattttttatttttttgagacggagtttcgctctttgttgcccaggctggagtgcaatggtgcgatctcagctcaccacaacctctgcttcccaggttcaagtgattctcctgtctcagcctcccaagtagctgggattacaagcgccagccaccacgcccggctaatttttgtatttttagtagagacagagtttcaccttgttagccaggctggtcttgaactcctgacttcaggtgatcttcccgccttggcctccctaagtgctgggattataggtgtgagccactgtgcccggcctgagccacggtgcctggcctggtcttatattaagaatacccaaaatgttcaactgaaatttgacatggcacaaacatttcaatagtctttttctcaaaaatgtaagtgtacttaaatattctaaaattataacttttcctataagtattgcataatcacaaaaacaaaaaatgcacttagtttttcgatgcaccaaaggatttatacagcctagccaatgcaggatattaaaggaaagagatgtggattggaagccacaggtccagatgagatggaataaagtgagaggagagcaggtctcctgaacacccttctgtcagggccaggaattgtgctatttccttctgtctcactacctccttcttccctcgaagtagagacactggcccagagcacttccagctgtatgataagcagtgtgttaaatgataaaaagcaaaggaaatcctaaaccctagtaccaccttaaatcatttgaaaatcatgtttcttgatttacctttctctctgacaaatttttaggactatgaagaactactaggaagacagaaattttaggatatttagggtgacaattagaagattaaggaaggcttttgagtataacagtagtccaaggaatcaaatgttcatcagaatccttattatggtggctcatgcctgtaaacccagcactttgggaggtcaagatgggaggatcacatagcttaggagcttgagaccacctaggcaacatagcgaaaccctgtctctactaaaaatgaaagaaaaattagcctagcatggtggttcctgccctgtagtcccagctactaaggaggctgaggatcacttgaacctgggagatggaggctacagtgagctataatcgcaccattgcaccccagcccaggcgacagagtgagatactgtgtcaaaaaaaaaaaaaaatccttttccccctctcattaacattcttttcactccctaatttctgaaagaactagatttttgaaagatgaaatatatgcttgaccagggcatgtaatgattagcagatcacagtatcatctcaacaacattcatgtggctgatgatctaaggcaagagaatgtaaagtagtcaaagtcacactatgtgcattttaagagacatactgcaccaaatgcaatagcgagcatggtctgcatccgggcatcttccagtgtgctcagtagcccttttttgctaagaagagctcttcctgccagggtccagaacttcacatgttttactcccactgagacaaactgggtatctgaatctggtcggaattctgccacaaaaatacgttgattgtgaccagctctgctggcaattttggcacctgacaagatacaacaaaattatctaggttattacaagaaccaagctaatcaacagcatcaaacaaatatgtaaaatacatagttcaaaaaacaaaggcttagaagagaggccaatggcccctgctctactacctagcaatacatgatttacaattatttgtgtattgagtccttttcacttatcttcgctccattaacttttctttatataacgtaaatgttttgtctaaagtgtggtaggtaatattatcctgctgatctgccattatcattagaaatatacataattttcataagaatctccaaaaccaatcaaatcattaataataaatacatagtttcttgctggaagaaaatagcagtgaatcatttataatgctaataatggtttcattaatttatctgttttgtgaggttacagttccactgggcttttaaagtgaaatatacctacagtaccactgtgtacagtatattgcataggcctccactgaatgattgtttcaaccaccaactttaagacaaatattaaatacagaattcctacta

SEQ ID NO: 77 - DNA, 23 bp, Artificial Sequence
  Synthetic construct
atgatgcaaagtgactaaaccag

SEQ ID NO: 78 - DNA, 27 bp, Artificial Sequence
  Synthetic construct
tctggtttagtacactttgcatcatat

(19) United States
(12) Patent Application Publication
(10) Pub. No.: US 2024/0002848 A1
(43) Pub. Date: Jan. 4, 2024

(54) COMPOSITIONS AND METHODS FOR SUPPRESSING MSUTZ

(71) Applicant: THE UNITED STATES GOVERMENT AS REPRESENTED BY THE DEPARTMENT OF VETERANS AFFAIRS, Washington, DC (US)

(72) Inventors: Brian Kraemer, Kent, WA (US); Jeanna M. Wheeler, Seattle, WA (US); Pamela McMillan, Seattle, WA (US); Timothy J. Strovas, Algona, WA (US); Jeremy Baker, Seattle, WA (US)

(21) Appl. No.: 18/038,307

(22) PCT Filed: Nov. 22, 2021
(86) PCT No.: PCT/US2021/060279

§ 371 Date: May 23, 2023

Related U.S. Application Data
(60) Provisional application No. 63/117,213, filed on Nov. 23, 2020.

(51) Int. Cl. C12N 15/113; A61K 31/55; A61K 31/27; A61K 31/445

(52) U.S. Cl. CPC C12N 15/113; A61K 31/55; A61K 31/27; A61K 31/445; C12N 2310/14

(57) ABSTRACT

Disclosed herein are small interfering RNA (siRNA) molecules and their use in methods and pharmaceutical compositions for inhibiting the expression of mammalian suppressor of tauopathy 2. Also, described herein are the use of said siRNA molecules in the treatment of Alzheimer's disease or dementia, and reducing accumulation of phosphorylated and aggregated human tau.

### CROSS REFERENCE TO RELATED APPLICATIONS

This application claims the benefit of U.S. Provisional Application No. 63/117,213, filed Nov. 23, 2020. The content of this earlier filed application is hereby incorporated by reference herein in its entirety.

### STATEMENT REGARDING FEDERALLY FUNDED RESEARCH

This invention was made with government support under grant number RF1AG055474 awarded by National Institutes of Health. The government has certain rights in the invention.

### INCORPORATION OF THE SEQUENCE LISTING

The present application contains a sequence listing that is submitted via EFS-Web concurrent with the filing of this application, containing the file name “37759_0352P1_SL.txt” which is 45,056 bytes in size, created on Nov. 19, 2021, and is herein incorporated by reference in its entirety.

### BACKGROUND

The molecular mechanisms underpinning neurodegenerative diseases include the cellular disruption of proteostasis. In Alzheimer's disease (AD), this disruption manifests as the deposition of amyloid plaques and neurofibrillary tangles (NFTs), the diagnostic pathological lesions of the disorder. While the mechanistic relationship between plaques and tangles remains unclear, abnormal tau and Aβ synergize to drive neurodegeneration in AD. A large body of evidence supports the idea of Aβ amyloid pathology initiating the disease process in AD. However, the discovery of tau mutations in frontotemporal lobar degeneration with tau inclusions (FTLD-tau) (P. Poorkaj, et al., Ann. Neurol. 43, 815-825 (1998); M. G. Spillantini, et al., Proc. Natl. Acad. Sci. U.S.A. 95, 7737-7741 (1998); L. N. Clark, et al., Proc. Natl. Acad. Sci. U.S.A. 95, 13103-13107 (1998); and M. Hutton, et al., Nature 393, 702-705 (1998)) demonstrates that tau pathology can cause neurodegeneration independent of amyloid plaques. Furthermore, tau pathology, not amyloid deposition, correlates with the severity of dementia in AD (L. M. Bierer, et al., Arch Neurol 52, 81-88 (1995). Thus, findings to date justify active investigation of the mechanistic underpinnings of both amyloid- and tau-mediated neurodegeneration in AD. Despite a diverse array of highly powered AD clinical trials targeting amyloid production, clearance, or deposition, none have been successful. Altogether, these observations suggest that tau-targeted therapies in conjunction with removal of amyloid may be required to achieve cognitive preservation when treating AD (M. R. Khanna, et al., Alzheimers Dement 12, 1051-1065 (2016); and C. Ballatore, et al., Nat Rev Neurosci 8, 663-672 (2007)).

### SUMMARY

Disclosed herein are compositions comprising a nucleic acid sequence or molecule wherein the nucleic acid comprises or consists of a sequence having the sequence set forth in:

(SEQ ID NO: 7)
UUUUCUGGUUUCUGUGCCACACUCAGU,
(SEQ ID NO: 9)
UUUUUCUGGUUUCUGUGCCACACUCAG,
(SEQ ID NO: 11)
GUUUUUCUGGUUUCUGUGCCACACUCA,
(SEQ ID NO: 13)
AGUUUUUCUGGUUUCUGUGCCACACUC,
(SEQ ID NO: 15)
AAGUUUUUCUGGUUUCUGUGCCACACU,
(SEQ ID NO: 17)
GCAGGCCAGUACUUGCAGCGCUCCAAA,
(SEQ ID NO: 19)
AAGCAGGGAAGUAACGGCAGAGCUGAC.
(SEQ ID NO: 21)
CAAGCAGGGAAGUAACGGCAGAGCUGA,
(SEQ ID NO: 23)
ACAAGCAGGGAAGUAACGGCAGAGCUG,
(SEQ ID NO: 25)
UACAAGCAGGGAAGUAACGGCAGAGCU
(SEQ ID NO: 27)
UUACAAGCAGGGAAGUAACGGCAGAGC,
(SEQ ID NO: 29)
CUUACAAGCAGGGAAGUAACGGCAGAG,
(SEQ ID NO: 31)
UCUUACAAGCAGGGAAGUAACGGCAGA,
(SEQ ID NO: 33)
UUCUUACAAGCAGGGAAGUAACGGCAG,
(SEQ ID NO: 35)
CACUCAUCUCAGCGUUAGAAAAGCUACC,
(SEQ ID NO: 37)
UCUGGUUUCUGUGCCACACUCAGUUCAC,
(SEQ ID NO: 39)
UACUUGCAGCGCUCCAAAAGUUUUUCUG,
(SEQ ID NO: 41)
UCCCCAUUUUUACAAGCAGGCCAGUACU,
(SEQ ID NO: 43)
GAUGGGGUGAUGGUAGGCACACUCAUCC,
(SEQ ID NO: 45)
UUGGGGAAGGCUUUGCAGGGUGAGAUGG,
(SEQ ID NO: 47)
AAACAUUUUUCAGCAAAUUUACAAUUGG,
(SEQ ID NO: 49)
UAUUUACAAUUUGGGUGAACAAACAAAC,
(SEQ ID NO: 51)
UCUGGUUUAGUACACUUUGCAUCAUAUU,
(SEQ ID NO: 53)
UACUCACAUGAGUGAAGGGACAAUCUGG,
(SEQ ID NO: 55)
UUGGAGACAGUACUGGAAUUCUUCUACU,
(SEQ ID NO: 57)
GUGGUGCUGGUGGUGCAACUGGUUUUGG,
(SEQ ID NO: 59)
ACGGCAGAGCUGACUACUGGAAGGUGGU,
(SEQ ID NO: 61)
CCAUCUUCUUACAAGCAGGGAAGUAACGG,
(SEQ ID NO: 63)
GUUUUGGAUGAUAGAAGGGACAUUCCAU,
(SEQ ID NO: 65)
UACAUUGAGUGUUAAACCUACAAUGUUU,
(SEQ ID NO: 67)
GUAGAAUGUGCAGUCCGGUCUUGUACAU,
(SEQ ID NO: 69)
UGGUGGGACAUUAAUGGUGGGAUGGUAG,
(SEQ ID NO: 71)
UCGAAUCCAUUUCAAGGCAUGUCGUGGU,
or
(SEQ ID NO: 73)
UUAUUCGCUGGUUUGAGGUCGAAUCCAU.

Disclosed herein are compositions comprising a nucleic acid sequence or molecule wherein the nucleic acid comprises or consists of a sequence having at least 90% identity to the sequence set forth in:

(SEQ ID NO: 7)
UUUUCUGGUUUCUGUGCCACACUCAGU,
(SEQ ID NO: 9)
UUUUUCUGGUUUCUGUGCCACACUCAG,
(SEQ ID NO: 11)
GUUUUUCUGGUUUCUGUGCCACACUCA,
(SEQ ID NO: 13)
AGUUUUUCUGGUUUCUGUGCCACACUC,
(SEQ ID NO: 15)
AAGUUUUUCUGGUUUCUGUGCCACACU,
(SEQ ID NO: 17)
GCAGGCCAGUACUUGCAGCGCUCCAAA,
(SEQ ID NO: 19)
AAGCAGGGAAGUAACGGCAGAGCUGAC.
(SEQ ID NO: 21)
CAAGCAGGGAAGUAACGGCAGAGCUGA,
(SEQ ID NO: 23)
ACAAGCAGGGAAGUAACGGCAGAGCUG,
(SEQ ID NO: 25)
UACAAGCAGGGAAGUAACGGCAGAGCU
(SEQ ID NO: 27)
UUACAAGCAGGGAAGUAACGGCAGAGC,
(SEQ ID NO: 29)
CUUACAAGCAGGGAAGUAACGGCAGAG,
(SEQ ID NO: 31)
UCUUACAAGCAGGGAAGUAACGGCAGA,
(SEQ ID NO: 33)
UUCUUACAAGCAGGGAAGUAACGGCAG,
(SEQ ID NO: 35)
CACUCAUCUCAGCGUUAGAAAAGCUACC,
(SEQ ID NO: 37)
UCUGGUUUCUGUGCCACACUCAGUUCAC,
(SEQ ID NO: 39)
UACUUGCAGCGCUCCAAAAGUUUUUCUG,
(SEQ ID NO: 41)
UCCCCAUUUUUACAAGCAGGCCAGUACU,
(SEQ ID NO: 43)
GAUGGGGUGAUGGUAGGCACACUCAUCC,
(SEQ ID NO: 45)
UUGGGGAAGGCUUUGCAGGGUGAGAUGG,
(SEQ ID NO: 47)
AAACAUUUUUCAGCAAAUUUACAAUUGG,
(SEQ ID NO: 49)
UAUUUACAAUUUGGGUGAACAAACAAAC,
(SEQ ID NO: 51)
UCUGGUUUAGUACACUUUGCAUCAUAUU,
(SEQ ID NO: 53)
UACUCACAUGAGUGAAGGGACAAUCUGG,
(SEQ ID NO: 55)
UUGGAGACAGUACUGGAAUUCUUCUACU,
(SEQ ID NO: 57)
GUGGUGCUGGUGGUGCAACUGGUUUUGG,
(SEQ ID NO: 59)
ACGGCAGAGCUGACUACUGGAAGGUGGU,
(SEQ ID NO: 61)
CCAUCUUCUUACAAGCAGGGAAGUAACGG,
(SEQ ID NO: 63)
GUUUUGGAUGAUAGAAGGGACAUUCCAU,
(SEQ ID NO: 65)
UACAUUGAGUGUUAAACCUACAAUGUUU,
(SEQ ID NO: 67)
GUAGAAUGUGCAGUCCGGUCUUGUACAU,
(SEQ ID NO: 69)
UGGUGGGACAUUAAUGGUGGGAUGGUAG,
(SEQ ID NO: 71)
UCGAAUCCAUUUCAAGGCAUGUCGUGGU,
or
(SEQ ID NO: 73)
UUAUUCGCUGGUUUGAGGUCGAAUCCAU.

Disclosed herein are siRNA molecules wherein the siRNA molecule specifically targets at least one sequence selected from the group consisting of SEQ ID NO: 1 to SEQ ID NO: 5, SEQ ID NO: 77 and SEQ ID NO: 78 and reduces expression of mammalian suppressor of tauopathy 2 (MSUT2) gene in a cell, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73.

Disclosed herein are methods of treating Alzheimer's disease or dementia, the methods comprising: administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, and wherein the therapeutically effective amount reduces accumulation of phosphorylated and aggregated human tau.

Disclosed herein are methods of treating Alzheimer's disease or dementia, the methods comprising: administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73, and wherein the therapeutically effective amount reduces accumulation of phosphorylated and aggregated human tau.

Disclosed herein are methods of inhibiting expression of a MSUT2 polynucleotide in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

Disclosed herein are methods of inhibiting expression of a MSUT2 polynucleotide in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73.

Disclosed herein are methods of reducing phosphorylated and aggregated human tau protein in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

Disclosed herein are methods of reducing phosphorylated and aggregated human tau protein in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73.

Disclosed herein are methods of suppressing expression of a MSUT2 polynucleotide in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

Disclosed herein are methods of suppressing expression of a MSUT2 polynucleotide in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73.

Disclosed herein are methods of potentiating a neuroinflammatory response to a pathological tau protein in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

Disclosed herein are methods of potentiating a neuroinflammatory response to a pathological tau protein in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73.

Disclosed herein are methods of decreasing astrocytosis or microgliosis in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

Disclosed herein are methods of decreasing astrocytosis or microgliosis in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73.

Disclosed herein are methods of reducing neuroinflammation in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

Disclosed herein are methods of reducing neuroinflammation in a subject, the methods comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73.

Disclosed herein are methods of inhibiting expression of a MSUT2 polynucleotide, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

Disclosed herein are methods of inhibiting expression of a MSUT2 polynucleotide, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73, wherein the siRNA molecule reduces accumulation of phosphorylated and aggregated tau.

Disclosed herein are methods of suppressing expression of a MSUT2 polynucleotide, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

Disclosed herein are methods of suppressing expression of a MSUT2 polynucleotide, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73, wherein the siRNA molecule reduces accumulation of phosphorylated and aggregated tau.

Disclosed herein are methods of potentiating a neuroinflammatory response to a pathological tau protein, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

Disclosed herein are methods of potentiating a neuroinflammatory response to a pathological tau protein, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73, wherein the siRNA molecule reduces accumulation of phosphorylated and aggregated tau.

Disclosed herein are methods of decreasing astrocytosis or microgliosis, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

Disclosed herein are methods of decreasing astrocytosis or microgliosis, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73, wherein the siRNA molecule reduces accumulation of phosphorylated and aggregated tau.

Disclosed herein are methods of reducing neuroinflammation, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

Disclosed herein are methods of reducing neuroinflammation, the methods comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence having at least 90% sequence identity to a sequence selected from the group consisting of SEQ ID NO: 6 to SEQ ID NO: 73, wherein the siRNA molecule reduces accumulation of phosphorylated and aggregated tau.

Other features and advantages of the present compositions and methods are illustrated in the description below, the drawings, and the claims.

### DETAILED DESCRIPTION

Many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

As used in the specification and in the claims, the term “comprising” can include the aspects “consisting of” and “consisting essentially of.” “Comprising” can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. The subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for Alzheimer's disease or dementia, such as, for example, prior to the administering step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment can also be administered to a subject to ameliorate one more signs of symptoms of a disease, disorder, and/or condition. For example, the disease, disorder, and/or condition can be relating to Alzheimer's disease, Alzheimer's disease-related dementia or dementia.

The phrase “nucleic acid” as used herein refers to a naturally occurring or synthetic oligonucleotide or polynucleotide, whether DNA or RNA or a DNA-RNA hybrid, single-stranded or double-stranded, sense or antisense, which is capable of hybridization to a complementary nucleic acid by Watson-Crick base-pairing. Nucleic acids as disclosed herein can also include nucleotide analogs (e.g., BrdU), and non-phosphodiester internucleoside linkages (e.g., peptide nucleic acid or thiodiester linkages). In particular, nucleic acids can include, without limitation, DNA, RNA, cDNA, gDNA, ssDNA, dsDNA or any combination thereof.

Nucleic acid sequences recited herein are written in a 5′ to 3′ direction unless otherwise indicated. The term: nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases these bases may appear as “dT” to differentiate deoxyribonucleotides present in a chain of ribonucleotides.

As used herein, the term “complementary” refers to the ability of a nucleic acid to form hydrogen bond(s) with another nucleic acid sequence by either traditional Watson-Crick or other non-traditional types. A percent complementary indicates the percentage of residues in a nucleic acid molecule which can form hydrogen bonds (e.g., Wastson-Crick base pairing) with a second nucleic acid sequence (e.g., 5, 6, 7, 8, 9, 10 out of 10 being 50%, 60%, 70%, 80%, 90%, and 100% complementary).

As used herein, the term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element or regulatory element). The terms “plasmid” and “vector” can be used interchangeably, as a plasmid is a commonly used form of vector. Moreover, this disclosure is intended to include other vectors which serve equivalent functions.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

Tauopathies are a heterogeneous group of neurodegenerative diseases characterized by abnormal metabolism of misfolded τ (tau) proteins leading to intracellular accumulation and formation of neurofibrillary tangles (NFT). In Alzheimer's disease (AD), tau neuropathology correlates with severity of dementia. However, interventions for AD and related dementias are limited to treatment of symptoms that do not directly alter tau pathology or the resultant neurodegeneration. This underscores the need for tau-targeted disease-modifying therapeutics. Furthermore, the results from amyloid-targeted clinical trials in AD patients suggest that achieving cognitive preservation in AD may require tau-targeted therapy in conjunction with the removal of amyloid. MSUT2 controls neuronal susceptibility to tau toxicity in the mammalian brain. The mechanism of MSUT2 modulation of tauopathy appears to involve MSUT2 binding to poly(A) RNA and its modulation of RNA polyadenylation. Described herein are siRNAs that inhibit MSUT2 from binding to poly(A) RNA providing a pharmacological means of intervening against tauopathy.

It has been shown that targeted reduction of the MSUT2 protein reverses the toxic consequences of pathological tau in animal models and human cells. Described herein are nucleotide sequences facilitating gene silencing approaches targeting MSUT2 such as RNA mediated interference and/or antisense oligonucleotides.

RNA interference (RNAi) is a naturally occurring post-transcriptional regulatory mechanism present in most eukaryotic cells that uses small double stranded RNA (dsRNA) molecules to direct homology-dependent gene silencing. Shortly after its first description, RNAi was also shown to occur in mammalian cells by means of double-stranded small interfering RNAs (siRNAs) 21 nucleotides long.

The process of RNA interference is thought to be an evolutionarily-conserved cellular defense mechanism used to prevent the expression of foreign genes and is commonly shared by diverse phyla and flora, where it is called post-transcriptional gene silencing.

The mechanism of RNAi is initiated when long double stranded RNAs are processed by an RNase III-like protein known as Dicer. The protein Dicer typically contains an N-terminal RNA helicase domain, an RNA-binding so-called Piwi/Argonaute/Zwille (PAZ) domain, two RNase III domains and a double-stranded RNA binding domain (dsRBD) (Collins et al. FEBS Letters, 2005, Vol. 579, Issue 26, pp. 5841-5849) and its activity leads to the processing of the long double stranded RNAs into 21-24 nucleotide double stranded siRNAs with 2 base 3′ overhangs and a 5′ phosphate and 3′ hydroxyl group. The resulting siRNA duplexes are then incorporated into the effector complex known as RNA-induced silencing complex (RISC), where the antisense or guide strand of the siRNA guides RISC to recognize and cleave target mRNA sequences (Elbashir et al. 2001, Nature, 411(6836):494-8) upon ATP-dependent unwinding of the double-stranded siRNA molecule through an RNA helicase activity (Nykanen et al. 2001, Cell, 107(3):309-21). The catalytic activity of RISC, which leads to mRNA degradation, is mediated by the endonuclease Argonaute 2 (AGO2) (Liu et al. 2004, Science, 305(5689):1437-41; and Song et al. 2004, Science, 305:1434-37). AGO2 belongs to the highly conserved Argonaute family of proteins. Argonaute proteins are about 100 KDa highly basic proteins that contain two common domains, namely PIWI and PAZ domains (Cerutti et al 2000, Trends Biochem. Sci, 25(10): 481-482). The PIWI domain is important for the interaction with Dicer and contains the nuclease activity responsible for the cleavage of mRNAs. AGO2 uses one strand of the siRNA duplex as a guide to find messenger RNAs containing complementary sequences and cleaves the phosphodiester backbone between bases 10 and 11 relative to the guide strand's 5′ end (Elbashir et al 2001, Nature, 411(6836):494-8). An important step during the activation of RISC is the cleavage of the sense or passenger strand by AGO2, removing this strand from the complex (Rand et al. 2005, Cell, 123(4): 621-9). Crystallography studies analyzing the interaction between the siRNA guide strand and the PIWI domain reveal that it is about 2 to 8 nucleotides that constitute a “seed sequence” that directs target mRNA recognition by RISC, and that a mismatch of a single nucleotide in this sequence may drastically affect silencing capability of the molecule (Ma et al. 2005, Nature 429, pp. 318-322; Doench et al. 2004, Genes Dev., 18(5): 504-11; and Lewis et al. 2003, Cell 115, pp. 787-798). Once the mRNA has been cleaved, due to the presence of unprotected RNA ends in the fragments the mRNA is further cleaved and degraded by intracellular nucleases and will no longer be translated into proteins (Orban et al. 2005, RNA, 11(4): 459-469) while RISC will be recycled for subsequent rounds (Hutvagner et al 2002, Science, 297(5589):2056-60). This constitutes a catalytic process leading to the selective reduction of specific mRNA molecules and the corresponding proteins. It is possible to exploit this native mechanism for gene silencing with the purpose of regulating any gene(s) of choice by directly delivering siRNA effectors into the cells or tissues, where they will activate RISC and produce a potent and specific silencing of the targeted mRNA.

### Compositions

Disclosed herein are target sequences and nucleic acids useful in the methods described herein. In some aspects, the target sequence(s) can be selected from one or more of the sequences listed in Table 1. In some aspects, the target can be MSUT2 gene (also known as ZC3H14). The mouse MSUT2 gene ID is 75553. The human MSUT2 gene ID is 79882. In some aspects, the target sequence can encompass a fragment of the mRNA MSUT2 sequence. In some aspects, the target sequence can encompass a fragment of the mRNA MSUT2 sequence, wherein the mRNA MSUT2 sequence comprises the ZF domain. In some aspects, the target sequence can be SEQ ID NO: 74 or a fragment thereof. In some aspects, the target sequence can encompass a fragment of SEQ ID NO: 75 or SEQ ID NO: 76. In some aspects, the target sequence can be SEQ ID NO: 77 or a fragment thereof. In some aspects, the target sequence can be SEQ ID NO: 78 or a fragment thereof. As used herein, the term “target sequence” as described herein is a target DNA sequence as used for definition of transcript variants in databases used for the purposes of designing siRNAs, whereas the specific compounds to be used will be RNA sequences defined as such.

A gene is “targeted” by a siRNA as described herein when, for example, the siRNA molecule selectively decreases or inhibits the expression of the gene. The phrase “selectively decrease or inhibit” as used herein encompasses siRNAs that affect expression of one gene, in this case MSUT2. Alternatively, a siRNA targets a gene when (one strand of) the siRNA hybridizes under stringent conditions to the gene transcript, i.e., its mRNA. Hybridizing “under stringent conditions” means annealing to the target sequence under standard conditions, e.g., high temperature and/or low salt content which tend to disfavor hybridization. A suitable protocol (involving 0.1.times.SSC, 68.degree. C. for 2 hours) is described in Maniatis, T., et al., Molecular Cloning: A Laboratory Manual, Cold Spring Harbor Laboratory, 1982, on pages 387-389.

In some aspects, the target sequence can encompass the MSUT2 ZF domain or a part or a portion of the MSUT2 ZF domain. The ZF domain is the functional part of the MSUT2 protein that binds poly(A) RNA. The short isoform of the MSUT2 protein encodes the ZF domain. The long isoforms of the MSUT2 protein can have additional domains. Targeting the other domains can allow the short isoform to continue carrying out the MSUT2 RNA binding function. In some aspects, to achieve a strong loss of function, the siRNA sequence can target the MSUT2 ZF domain.

In some aspects, a target sequence described herein can comprise or consist of at least one sequence selected from SEQ ID NO: 1 to SEQ ID NO: 5, SEQ ID NO: 74 to SEQ ID NO: 76, SEQ ID NO: 77, and SEQ ID NO: 78.

TABLE 1
Examples of Target Sequences

| | | | SEQ |
|---|---|---|---|
| Target | | | ID |
| Gene | Name | Sequence | NO. |
| MSUT2/ | crRNA_human | 5′-AATTTATCGACCACCTGCAAG-3′ | 1 |
| ZC3H14 | and mouse | | |
| | MSUT2 E6 | | |
| MSUT2/ | crRNA_mouse | 5′-TACTGGCCTGCCTGTAAAAAT-3′ | 2 |
| ZC3H14 | MSUT2 E13 | | |
| MSUT2/ | crRNA_mouse | 5′-GGCCTGCCTGTAAAAATGGGG-3′ | 3 |
| ZC3H14 | MSUT2 E13 | | |
| MSUT2/ | crRNA_mouse | 5′-GCCACCAAGACACGCCTTGAA-3′ | 4 |
| ZC3H14 | MSUT2 E16 | | |
| MSUT2/ | MSUT2 | 5′-ATTAGACACTTCAGATAGAT-3′ | 5 |
| ZC3H14 | sgRNA | | |
| | 3′UTR#1 | | |
| MSUT2 | ZF Domain | 5′-GGTAGCTTTTCTAACGCTGAGAT | 74 |
| | | GAGTGAACTGAGTGTGGCACAGAAAC | |
| | | CAGAAAAACTTTTGGAGCGCTGCAAG | |
| | | TACTGGCCTGCTTGTAAAAATGGGGA | |
| | | TGAGTGTGCCTACCATCACCCCATCT | |
| | | CACCCTGCAAAGCCTTCCCCAATTGT | |
| | | AAATTTGCTGAAAAATGTTTGTTTGT | |
| | | TCACCCAAATTGTAAATATGATGCAA | |
| | | AGTGTACTAAACCAGATTGTCCCTTC | |
| | | ACTCATGTGAGTAGAAGAATTCCAGT | |
| | | ACTGTCTCCAAAACCAGTTGCACCAC | |
| | | CAGCACCACCTTCCAGTAGTCAGCTC | |
| | | TGCCGTTACTTCCCTGCTTGTAAGAA | |
| | | GATGGAATGTCCCTTCTATCATCCAA | |
| | | AACATTGTAGGTTTAACACTCAATGT | |
| | | ACAAGACCGGACTGCACATTCTACCA | |
| | | TCCCACCATTAATGTCCCACCACGAC | |
| | | ATGCCTTGAAATGGATTCGACCTCAA | |
| | | ACCAGCGAATAA-3′ | |
| MSUT2/ | Standard | 5′-ATGATGCAAAGTGACTAAACCA | 77 |
| ZC3H14 | MSUT2 RNAi | G-3′ | |
| MSUT2/ | Standard | 5′-TCTGGTTTAGTACACTTTGCAT | 78 |
| ZC3H14 | MSUT2 RNAi | CATAT-3′ | |

```
The mouse longest coding mRNA → protein is: NM_029334.2 → NP_083610.2
(SEQ ID NO: 75)
        1 ggggacgcgc acggcggagg cggagcggcg gcggcagcgg cggcagcggc agcggcagcg
      61 gcgtaggggg cccaggctgc agggtggcag cccgcggcgg gctccaggta accgaggcgc
    121 cgcgcagtgc cgagccggcc gcccgccgcc gagccatgga aatcggcacc gagatcagcc
    181 gcaagatccg gagtgccatt aaggggaaat tacaagaatt aggagcttac gtagatgaag
    241 aacttcctga ttacattatg gtgatggtgg ccaacaagaa aagtcaggac caaatgacag
    301 aggacctgtc cctgtttcta gggaacaaca caattcgatt caccgtatgg ctccatggtg
    361 tattagataa actgcgctct gtcacgactg agccctctag tctaaagtct cctgacgcca
    421 gcatcttcga tagtcacgtg ccttcaaaca agagcagttt cagtcgggga gatgagagaa
    481 ggcacgaagc tgccgtccct ccccttgctg tttctagttc tagacctgaa aagagggatt
    541 ccagagtttc tacaagttca caggagcaga aatccactaa tgtcagacat tcatatgatg
    601 atggagcttc cacccggcta atgtcaacag tgaaacctct gagggaacca gcaccctctg
    661 aagatgtgat tgatatcaag ccagaaccag atgatctcat tgatgaagac ctcaattttg
    721 tgcaggagaa tcccttatct cagaaaaaac ctacagtgac acttacatac ggttcttctc
    781 gcccttctat tgaaatttat cgaccacctg caagtagaaa tgcagacact ggtactcact
    841 taaacaggct gcaacttcat ccgcagcaaa gcagtgctca cgctgccaag cagctggatg
    901 tacaaagcag ccaggtatcc gaagcaggac ggttgtgtga gccaccagtg cttagcagcg
    961 tagaagacac ttatagcccc ttcttcagaa acaacttgga taaaatgagt attgaggacg
  1021 aaaactttcg aaagagaaaa ttgcctgtgg taagttcggt tgttaaagta aaaagattta
  1081 gccatgatgg agaagaggag gaagaagatg aggattatgg gacccgcata ggaagcttgt
  1141 ccagcagcgt gtcagtacca gcaaagcctg agaggagacc ttctcttcca ccttctaaac
  1201 aagctaacaa gaatctaatt ttgaaggcta tctctgaagc tcaagagtct gtaacaaaga
  1261 caactaacta ttctgcagtt ccacagaaac agacacttcc agttgctccc agaactcgaa
  1321 cttctcaaga agaattgcta gcagaaatgg tccaggggca aaacagggcc cccagaataa
  1381 gtccccctgt taaagaagag gaagcaaaag gagataatac aggaaaaagt caaggaactc
  1441 aacagaggca attgttatcc cgactgcaaa ttgatccagt aatggtagaa acaatggaga
  1501 tgagtcaaga ttactatgac atggaatcca tggtccatgc agacacaaga tcatttattc
  1561 tgaagaagcc aaagctgtct gaggaaatag tagtgacacc caaccaggat tcggggatga
  1621 agactgcaga tgcccttcgg gtcctttcag gacaccttat gcagacacga gatcttgtac
  1681 aaccagataa acctgcaagt cccaagttta tagtgacgct ggatggtgtc cccagccccc
  1741 caggatacat gtcagatcaa gaggaggaga tgtgctttga aggaatgaaa cccgtaaacc
  1801 aaacttcagc ctcaaacaag ggactcagag gtctcctcca cccacagcag ttgcatttgc
  1861 tgagcaggca gcttgaggac ccagatggta gcttttccaa cgccgagatg actgacctga
  1921 gtgtggcaca gaaaccagaa aaacttctgg agcgctgcaa gtactggcct gcctgtaaaa
  1981 atggggatga gtgtgtatac catcatccca tttcaccttg caaagccttt cccaactgta
  2041 aatttgctga gaaatgtttg tttgtgcatc caaattgtaa atatgacaca aagtgtacta
  2101 aagcagattg tcccttcact cacatgagta gaagagcctc gatactgact ccaaaaccag
  2161 tgtcgtcacc agcaccgtct tctaatggcc agctctgccg ttacttccct gcttgtaaga
  2221 aaatggaatg tcccttctac cacccaaaac actgtaggtt taacactcag tgtacgagac
  2281 ctgactgcac attttatcac cccaccatta ctgtgccacc aagacacgcc ttgaaatgga
  2341 ttcgacctca gagcagtgag tgatgcccta gtcctacctg gcagaagatc atgcagtttg
  2401 aaagcttcca tcttctgatg agagatgttc tacagaactt gtcacgtctt tgaaatttag
  2461 aatatattgc tttcataata cgaattttac tgccccactg aagtgtctaa tttttcaagt
  2521 ttgtaagttt attaagtggt ttcaacattt tttgtttgtt cgttttgact atgaaaaaga
  2581 cagtttaaag aaaagccaaa ttctattaaa acatttgcgg catgtttgta cattgctgtt
  2641 taatatcatt tttggtaatg gtacttgcag cttagggctg tagtgctgtg ggaaggccag
  2701 tgtcctcaga gctgaagcac ttttcagctt ttcccaaagg taatgcagtg tctgtaaccc
  2761 agcgtggtaa cagtggccag gctttgaaac tgaggcagct ttggaacaac tagtttaaat
  2821 ttcttttttt agtgtctaaa tgaatttgct ctgagaagca taatgcagac tttattttga
  2881 gtgctacttt ggtagagtgg accgaggtcc tgtgcctttc tgaaagtgag cagagacatg
  2941 gtcataaagg gtaagcatag ttggaatgac gatgtaaaaa tatatggaca gttctttgga
  3001 atgctcccat ttactattag cttatcattt tataagtaat tttggaggga ctacattatc
  3061 acaaaagtat acaaaaattt ttacaggcat atgtacagaa agtatcagaa aacagacttt
  3121 gaactcacaa gaatataaat atacgtatat attcccatat tctgaaaaat atcatcagaa
  3181 ataaccccac agaaaatata cttatgttat tactaaagat cattcttgaa atgtagaagt
  3241 tgagatttaa gtggtatatt ttaaatgaca gaactatatt gcagagatag gaaggtaaac
  3301 ttgacaatag gatgaaactt ggcctactgt actatggagt tttatgtgtg gtttttgaaa
  3361 ctgttaaggc aagatgtgtc atgttttaga actaaataac agacaactga tttcaaaaac
  3421 gtgttgtttt aaaaattaaa gtgtaaacgg tggttagcaa aggggataat aaaagctcaa
  3481 acattttgag gaccaaattt aactgttaag atacaataaa gtcacatcta taaaagtctg
  3541 tgtttaataa tgtgaa.
The human longest coding mRNA is: NM_024824.5 → NP_079100.2
(SEQ ID NO: 76)
        1 ggaggcggtg gtgtcccggc tgcggggtag gagtccgcgg cagcctccgg gtaagccaag
      61 cgccgcgcag tgctgagttc ccgcacgccg cagagccatg gagatcggca ccgagatcag
    121 ccgcaagatc cggagtgcca ttaaggggaa attacaagaa ttaggagctt atgttgatga
    181 agaacttcct gattacatta tggtgatggt ggccaacaag aaaagtcagg accaaatgac
    241 agaggatctg tccctgtttc tagggaacaa cacaattcga ttcaccgtat ggcttcatgg
    301 tgtattagat aaacttcgct ctgttacaac tgaaccctct agtctgaagt cttctgatac
    361 caacatcttt gatagtaacg tgccttcaaa caagagcaat ttcagtcggg gagatgagag
    421 gaggcatgaa gctgcagtgc caccacttgc cattcctagc gcgagacctg aaaaaagaga
    481 ttccagagtt tctacaagtt cgcaggagtc aaaaaccaca aatgtcagac agacttacga
    541 tgatggagct gcaacccgac taatgtcaac agtgaaacct ttgagggagc cagcaccctc
    601 tgaagatgtg attgatatta agccagaacc agatgatctc attgacgaag acctcaactt
    661 tgtgcaggag aatcccttat ctcagaaaaa acctacagtg acacttacat atggttcttc
    721 tcgcccttct attgaaattt atcgaccacc tgcaagtaga aatgcagata gtggtgttca
    781 tttaaacagg ttgcaatttc aacagcagca gaatagtatt catgctgcca agcagcttga
    841 tatgcagagt agttgggtat atgaaacagg acgtttgtgt gaaccagagg tgcttaacag
    901 cttagaagaa acgtatagtc cgttctttag aaacaactcg gagaaaatga gtatggagga
    961 tgaaaacttt cggaagagaa agttgcctgt ggtaagttca gttgttaaag taaaaaaatt
  1021 caatcatgat ggagaagagg aggaagaaga tgatgattac gggtctcgaa caggaagcat
  1081 ctccagcagt gtgtctgtgc ctgcaaagcc tgaaaggaga ccttctcttc caccttctaa
  1141 acaagctaac aagaatctga ttttgaaggc tatatctgaa gctcaagaat ccgtaacaaa
  1201 aacaactaac tactctacag ttccacagaa acagacactt ccagttgctc ccagaactcg
  1261 aacttctcaa gaagaattgc tagcagaagt ggtccaggga caaagtagga cccccagaat
  1321 aagtcccccc attaaagaag aggaaacaaa aggagattct gtagaaaaaa atcaaggaac
  1381 tcaacagagg caattattat cccgactgca aatcgaccca gtaatggcag aaactctgca
  1441 gatgagtcaa gattactatg acatggaatc catggtccat gcagacacaa gatcatttat
  1501 tctgaagaag ccaaagctgt ctgaggaagt agtagtggca ccaaaccaag agtcggggat
  1561 gaagactgca gattcccttc gggtactttc aggacacctt atgcagacac gagatcttgt
  1621 acaaccagat aaacctgcaa gtcccaagtt tatagtgacg ctggatggtg tccccagccc
  1681 cccaggatac atgtcagatc aagaggagga catgtgcttt gaaggaatga aacccgtaaa
  1741 ccaaactgca gcctcaaaca agggactcag aggtctcctc cacccacagc agttgcactt
  1801 gctgagcagg cagcttgagg acccaaatgg tagcttttct aacgctgaga tgagtgaact
  1861 gagtgtggca cagaaaccag aaaaactttt ggagcgctgc aagtactggc ctgcttgtaa
  1921 aaatggggat gagtgtgcct accatcaccc catctcaccc tgcaaagcct tccccaattg
  1981 taaatttgct gaaaaatgtt tgtttgttca cccaaattgt aaatatgatg caaagtgtac
  2041 taaaccagat tgtcccttca ctcatgtgag tagaagaatt ccagtactgt ctccaaaacc
  2101 agcagttgca ccaccagcac caccttccag tagtcagctc tgccgttact tccctgcttg
  2161 taagaagatg gaatgtccct tctatcatcc aaaacattgt aggtttaaca ctcaatgtac
  2221 aagaccggac tgcacattct accatcccac cattaatgtc ccaccacgac atgccttgaa
  2281 atggattcga cctcaaacca gcgaatagca cccagtcctg cctggcagaa gatcatgcag
  2341 tttggaagtt ttcatgtact gatgaaagat actctacaga acttgtcaaa tctttgaaac
  2401 ttggaatata ttgctttcat aatatgaagt tttattgcct atctatctga agtgtctaat
  2461 ttttcaagtt tgtaagttta ttatgtggtt ttaacattgg gtgtttttgt tttgttttta
  2521 ctatgaaaag acagcttaag gaagagctaa attctgttaa aatatttggg gcatgtttgt
  2581 gcactgctgt tgtgaggatc agcatatgaa attgacatca tggttagtca tggtactgca
  2641 gcttaggggg ctacacggtt gctgtgtgag tggagagatg cagtgaggca gttgtcatta
  2701 ttctaaaaat tgtactactt tcacttttcc caaagattat ataatgttca taatccacca
  2761 tgaaaacagc attggccaaa ggtactgagg ctgcttaaaa tattcaattc tgctttttaa
  2821 tttttaagtg aatttagttt gaaaagcatg attatacagg cctctcaggc tgagtgctac
  2881 tttggtaaag ttcccagttt tcctgccttc tgtgacagga tgaatgaggt gggtatggac
  2941 agtggaggca gctggaatgg caagtgcaga aaataggaac agttctatac agtgctctca
  3001 tttactaata acataatgcc ttctaaataa tttttttggg aaactacatt atcacaaaat
  3061 tatacaaatt tttttacaag tatttacata ctgtatctga aaacagactt taaagtcaca
  3121 agattataaa tgtacatata tattctcaca ttctgaaaaa taacattctc agaatccaca
  3181 gaaaatatac ttagttacta ctgaagataa tttttgaaat gtaaaaatta gatttaaata
  3241 gtatatttta aatgacagaa ctataattac agagatcaga tcagataggt aaactgcaag
  3301 atagatagga tgaaactttt ggcctactgt attacttaca gagttttttt gtgtgtggtt
  3361 tttaaaactg ttaaggcaag aagtgtcaaa tgctttagag ttaaataaca gatcactgat
  3421 ttcaaagact tggtgtatag tgttaaaaat taaagcttaa aaggtggtta gaaaagtgga
  3481 ttaatgcaaa aggggtaata aagactgcaa cattctcagg accaaattaa actgctaaaa
  3541 aaaaaaaaaa agttcattga cttgcttagt cgtatactca aatgatgata aacctacatg
  3601 tgcaaaggct cacgtttaag attgtcaagc cagcagtcta ctgttgtgtt gccattgctt
  3661 ttccattggg agaagaaaga attaaccagt cattaaacca tttggtaagt tgcactttgc
  3721 tgtgctgatc ccacaggaaa ggcttgaaac acgagaagca gcaaagacag agcacacaag
  3781 tgcataaggc tgttgtcttc ggcttgggtg aaatgacagt tcctcttcat tctaaaggtt
  3841 tactccattg aatttaaggc atttgttcat tccagtgttg agatgctttg catctctgca
  3901 gaagaaattt attttaaatt gtttaaatat ctggaaatac ttttagctat catttataaa
  3961 gatagttttg ttctcagttt cactataaat tatagaacaa atgggaaaca agggtttaat
  4021 ttagttcagc cattttacaa ggaaataata aaatactaaa atctgattgt tttttgctat
  4081 ttaatagcca ctgcccagac acatatttaa gagtttaatc tttcagttgc tatggcttat
  4141 gaacaagcta aggttgacca taaaacattt gttggatgac gtggtttaaa atgatcacca
  4201 caaaaaggga ccacaaaaaa aggaaggaaa tgagcatggt tggcgattgg aagcaagggt
  4261 accagagggc acagtgtgct ttggcatgca ttttatacat aaaatgaatg gaacaaaagg
  4321 tgccagaagt cccaggttac acaatcagga gcttagatac tgcacacaaa aataattatc
  4381 tgggttaaaa aagtaaacat agggcagatt ctatatggcc tatcatgttt cttcaccttc
  4441 ccctcgttgc tggctgatac agcgaggtgg tcagctgatg actacttagt caatatgacc
  4501 tttagtcgtg aaactgacag cagcagtgat taaggctgac ttaatcaggt tggccacttt
  4561 gaaggacaga aatgcagtgg aaacagtttt attctatgta gtttacatgc ttaaggttac
  4621 agagtttcta cctgcactgt aatggaaata taatttctct gtagccaaaa gctggcaaac
  4681 ttgacccaga gggaaaattt aaaactgcag caggctcaaa tgtagagtat ttttcttttt
  4741 atgggcaggt tgttcaggga tttttttcct cctttaattt attgactgac tgtaaataca
  4801 tgagtagaaa cttaatagtc atgtatttca aaatttggct taatttagga gaatccactg
  4861 atgaacaagt accaacttac gtttcaagct tcttagcccc ataatcagtc cttcagccac
  4921 agctatttag agctttaaaa ctaccaggtt caatcactgg ttatgctttc tgtgatgtaa
  4981 tttagtcatt tctattttta gtattaacca agtattagac acagaaaata ggtattaaga
  5041 atcttcatat atcctgtcag accaaatggg attccaggaa cctaaagcga tctattatgc
  5101 tataaagata attaacacat taaaaactca tagggtcaat acagcatctt aaacctcaca
  5161 cttagaaaaa tatattttta aatagcagtc tacataattt tcaatcttca ggaaactaca
  5221 gataggctag acagcgaatt cctgaatgat gagtagtgat ctttggcagc atttaaagtg
  5281 aaaagaaata aggatctaag aattcagccc taatccacta aaaaaaggaa ttctaactga
  5341 caagttttta caaatggagt tgggctcatt cattttggaa ataaacctat ggagtggcac
  5401 acatctaaac aaattttccc aatagaaaaa aggctataaa aattttattc caagagtgat
  5461 taaattgtat aatgttgtat atgtgaattt aacacttttg tttacatgtt aaacaaatgt
  5521 gtatatatta gactacatta aatatgcaat tctttcttcc agttaaatac tgttgctccc
  5581 taaaaccctt acattgtaca ccattgggaa tgattgttca tcatactact tttccattag
  5641 tgaggctaca gttatgtttt aaatgtgcga ttacagagat ggcatctgaa cataaactga
  5701 tggctcgaaa atgaaaatgg aaatgtagca gccatatact gctaactttg gatctgttcc
  5761 tgaattcaaa actactagga gaaaagtgtc ctttataaaa aaggacctta ttaatgccta
  5821 aaaaacatca tattctctag gaaagcttgt gtctgtttcc ttagggaaaa tgtttgcctt
  5881 ttaaaaactg tgatccttta ggatgatcat gactttccct ttccttatgg aaatgcaaga
  5941 ataaaatatt tcattaaaca atgaaccttg aaaataaaat ataaacatta agaaaccatt
  6001 ttgctaaaaa gataatgaaa attatccaaa ttgggttttt gagttcttct gtaaagagtg
  6061 ctctacccta aattttccca gcaggtctgc cgaaatcaca cacttcccaa tacaggggga
  6121 cttggccttt accatcaagt attcgatcct tccttgaaat ggcattatct ggcagtgtat
  6181 ggattacgga ttatacccag tgcatatagc aaatattttg aacagatcag tctttcacta
  6241 ttttgatgat tctgggcatt tctccctgtt acagtcttgg gttagcacca cttgaccatg
  6301 cagggttggg ttttggtttt tcttctctgt aattctggtc tcaaagttaa tttctgtagt
  6361 catctcagca tctctcagtg aggtgtatgt acacatttcc agacaaataa gctgcaatca
  6421 gagaagaaaa ttgcagggag ttaattatgt ttttagattt tcataacagt ttaatatttt
  6481 tcagttgtgc tttcaggtta catgtgtaat atttttcctc tttaactcct tttattctgt
  6541 atttgcataa atatgagatt ctgaagagcc atctggttat actaccttct actaatgttg
  6601 actagctgat ttcataaacc aaagctgtag gagttgttgt attaagtctc ttaactagta
  6661 acatagtctg ctcttcatgg gctgagaaag ttactaacct gcagtcatca cctccagcac
  6721 taacaacatg tcgatcacca ctggtaaatc gaatatttgt cacatggggc gaatgaccca
  6781 agaacctttt gtgttttgcc taaaaaacaa tgacagacaa gctcagggca tttggtgcac
  6841 acagaagtca aaggctctta ttaggaacta taatctctat gacaagagct gtggagagag
  6901 tagggagtta gcaccgcagc cagtgattag aatgcttttc agcatgagta gtggatctgc
  6961 aaaaccaggc tgtgtgggca gtcagatgtc tccaggtact ctgaccattt ttctctaagg
  7021 aaaagcattt gaaatttgat aactgattat aggtttggtg aaaagctaat tacagctttt
  7081 gtaggatggt tccaaagatg gtattactcg agggagagga tttgtttcta atagctttta
  7141 tttcaaagta aatagattta gaaagtttgg ggaaaaattt agaaattagg acaaaacatt
  7201 ttaaatatat ggggaaaagt gctgatgata agacatcaaa attaggagta aactgataat
  7261 agtaaacaaa acacaaactt acaaattttt ctggacatgg gaagtcaaat aacttaacca
  7321 tgccaaagtc atctcctgta acaagactga ttcctgaatg agatacacag gcacagttga
  7381 catcagcttt ctcagcatgt ctggaccaga ttcccaaaac ctcatctcct agaatactag
  7441 agggaaggaa caaaagaaaa ctcatcatgg caagtgcggg caggttgact atattcaaaa
  7501 agtttcttgg caattaatct ctaagtaccc tatcatgtta cttaaaatac aggaagtaaa
  7561 ttatggtaag ttgtttggag acctgaattt catcaggata tcaactcctg ccttttaaaa
  7621 atgacatttt ataatttgaa gggtttctag attaatcttt ttaagattaa agtagtactt
  7681 tatgaaaact gatagaacta ttttttcttt tttttttttt gagacggagt tttcgctctt
  7741 gttacccagg ctggagtgca atggcatgat ctcggctcac cgcaacctct gcctcctggg
  7801 ttcaagcaat tctcctgcct cagcctcccg agtagctggg atcacaggca tgcgctaaca
  7861 tgcccggctt attttgtatt tttagtagag acagggtttc tccatgttgg tcaggctagt
  7921 ctcgaactcc cgacctcagg tgatcacccc cgctcggcct cccaaagtgc tgggattaca
  7981 ggctgagcca ccgcgcctga ctgaaaactg atagaactat ttttcaaatt aaaagtgcta
  8041 cttggctggg tccagcagca cataccagta atcccaacat tttgggaggc tgaggcagga
  8101 ggactgcttg aggccaagag tttgagacca gcctgggcaa tattgtgaga tccctatctc
  8161 tacaaaaata aaaatgactt atgacatagg aattaaaaaa atttcagaga tggggtcttg
  8221 ctatgttgcc caggctggta tcaaaacttc taggctcaag tgatcctccc acctcggcct
  8281 gctacatcag agattacagg catgagccac tatatgcctg gctgatacag gaatttgatg
  8341 gcatttttca ttggccaaaa aaatggatag tcatggttac ctgtcataca gccaggaaat
  8401 ttgaacaaat ttggaagctt tgacttctaa tagattcaag atagcattcc tttagataga
  8461 gaattaataa cagttgctta acagcaccca ataccttttt gccagtcatt aaatttagca
  8521 ttaagaaaaa tatcagggta tctttaaagt taaaactttg atttccttaa aaaaaaaact
  8581 tgataaatca tggaaactga taaaacatgg aaatatattc aataaaaagg ggtcccaaca
  8641 tgaacatacc atttcaaaat atggtaacaa aaacttgaaa ctcaattact attccttatt
  8701 ggaatggctc taacagttca gaaataggat tttctaactg gccttcaaag tcagttcttg
  8761 ccttgtgaat atataagtat ttacctagtc catgtagccc aagtaattct gtcaatagcg
  8821 gcatgatcca taagatgttt tcctgaaggc acttcataga catgccgttt atagcagcca
  8881 ctagagacct ttttcatcag attaaaatgg gacaagaatt ccattaggtg agagacaaaa
  8941 tccacagggg gtttacagaa tactagcata ttgctacttg atttacatgt ctaacattat
  9001 taagtatgca aaagatcact acaaaaactt aataggagaa aagctctgat aagtggggga
  9061 ggaaagggga gctgtaggtc agaaggtaca aagggaggag ttgagaagct ggagctctgg
  9121 agctcaggaa ctttaaatgc attcactaac acgaaatgta aaagcagaag aacttgccac
  9181 ctgggtatac agtattggta ctgtacctgg agataactgc tatctgcaga gaagtccatt
  9241 tgaatgacaa agcttggaat gtctttgcag tagctgattc tgttaagagt ggggcccagc
  9301 gttaggtcat aaaaatccac tgagttctca ctagaaccta ctgccagata ccgggaatcc
  9361 ggactaaatc tgaatcaaaa caaaacgtaa aaagtattag accacatgaa gtattataaa
  9421 tacttaagat cagtgacttt tcctttctag ttcttaaaag taacgtgtga taaggcctca
  9481 aatagattta cctgtcagac acaactgatc atgtatactg agattgtctg ggttacatga
  9541 aataaggaag ctttatattt tacttaaatt ttaaatattt ccccaattgt catctcccaa
  9601 ttcctttaaa aacgtctaat ggcttaaaaa aactttctta ggccaggccc agtggctcac
  9661 acctataatc ccagaacttt gggaagcgga ggcgggcaga tcacctgagg tcgagagttt
  9721 gagaccagcc tgaccaacat agagaaaccc tgtctctact aaaaatacaa aattagccag
  9781 gcatggtggt gcacgcctgt aatcccatct actcgggagg ctgaagcagg agaatcgctt
  9841 gaacccagga ggcacaggtt gtggtgagct gagattgcac cattgcactt cagcatgggc
  9901 aacaagagca aaactccaac tcaaaacaaa acaaaacaaa atttaatttt ttaaatagag
  9961 gcggggtctc actatggtcc caaactcctg gcctcaagca atccttcccc cttggcctcc
10021 caaggtactg ggattacagg tgtgagccac aacacccagt cagaacatct cagcttttaa
10081 aagccattag cattacataa ttaataagct aacaattcat taagatagtt ttcttccatc
10141 tggaaaaaac gttgtcttaa tattaagcaa agaacacagc ccagcttaac taacctccag
10201 ttattaaggt gaaatgacac aacttgaatc ttggaagaag aatttttttt ttttgagacg
10261 aagtctcgct cttgtctccc aggctggagt gcgatggcgc aacctccgcc tcccgggttc
10321 aagcgattct cctgtttcag ccccctgagt agctgggatt acaggcgcct gccaccacgc
10381 ccggctgatt tttgtatttt tagttgagat ggggtttcac tatgttggcc aggctggtcg
10441 agtactcctg acttcaggtg atctgcctgc ctcggcctcc caaagtgctg ggattacagg
10501 catgagccac cgcgcccggc ctgaagaact tatttaaaag acaaagtgaa atgctatttg
10561 cctagcaatc tttggagtca tatgggacaa ttcagtctct tgaaatggcc catgagtctt
10621 actgaggtac gatagagaca tgtaaaagct aagggaagcc actgttacta ttttatatat
10681 tgaagttctg aggaaggttt catttgtaaa aggattttac tgatgaaaag tgtacaagct
10741 tttgacagac ctagattcaa taatcttatc tactgatcac acggaagtac tccgtaaatg
10801 gtagccactg ttgaaaaatg cttaagcact gaaaaacaaa ggtttaagaa acatttaaat
10861 taatttggat tctggaacat ttaatcaata ggtattgatt aaattaatga actacatatt
10921 cccaaactga ggttactaag agaagatatg tttgaaatca caactttagt tttccagggt
10981 gacaactttt gaagggcaga tagctctctt gtattacagt gggagatacc tcttggtggg
11041 atgaacttaa tggacatggc taagtgttaa catgaattca tcaaacatta cctactagta
11101 cttgctatta tagttggtgc ccagtgggtt tataatttag caagaagaat taagtagtat
11161 acaaacagcc atattttagc atacaattta taatacggga aatgctacag gccctgggga
11221 cctctttttg aaggcaaggc tatggaaaat tttacaaatg gaagttaaat caagtatata
11281 ctagaaactc tattccattt gttcactaac ctgatatcat ggattgcaca tctcctgtct
11341 ctcttctttc cccatatttt tagagaactc actagtaaaa tgataaattc tccatttttc
11401 attccaatag ccaccatgtc cccttcaggg ctgtaacaca cagtacgagc agcatgtccc
11461 aaattcactt tgtttaacat cttctgcatt taaaaaaaaa aaaaaaaaga gtcataggaa
11521 acattaagtg aagtacttct aaattatacc agtttcccct caaaatgctc aacagaattc
11581 tggcagttct ttaagtacta gcaatttaga acttccaact tttcttttta gaagttgtaa
11641 cctcttttaa aaaaattatc tgtacttact ttatcagcaa tatcccaaag tctcactgtc
11701 ccatcttctg cagcagaaag gaaaaaatcc ctggaaggat gtgttgctag tccccagatt
11761 ggcccatcca catgaccgtt aactaaaata ttacaagctg catttttctc tccaacttcg
11821 attatttcag cattccttgt cccaacaagg atcttgccct gaaacacaag caggaccaat
11881 acagtgaatg taatacaaca gctgcttttc ttcttcataa tataaaaatg accctattga
11941 cctgctttca gagaactttt tgctttgagc taatctagta gcaaggcagt cattagctca
12001 tgcaaatttt tctatgacta caggcacaca tctatctgta agcacaatgg gctagattac
12061 atattagagt ccatgctaca gaatagaact tttctgtggc agtacacctg gattcttcaa
12121 taatcaaagt ttttatttga taatcttagg atttccaaac tggggtcagt gcagtgggat
12181 ataggaaaaa ataatagaat ttatttttta gttaaaaagt aaaagcttaa ctacaattta
12241 atatgcaggc tgaagataat atccgtatga tttataaata cacttaataa gtacaaacac
12301 gctcaaaaat tttcatagga gttgtagttt tgaattttta ttttgaaatt gacacataat
12361 tatacatatc tatagggcat agggtaatac gcataaccat cacctcagac atttatcatt
12421 tctttgtgat ggaaactttc aaaatcctct cttgtaaata cctgaaaata cataaatacg
12481 tgattcttaa ctatagtcat cctacagtac tacagaatac taaaacatac tattcctatc
12541 tggctgtgta aacttgtatc ctttaaccag tccttcccta tccccctccc cctccccctt
12601 gtccgcctcc agtaaccact attctactct ccacctctgt gggatcaact tttttagttt
12661 ctgcacagga gtgagaacat gtatttatct ttctgtgcct ggcttatttc acttcacatc
12721 atgtcctcca gtctcatcca tgttgccacc aagaatgaca gaatttcatt attttttatg
12781 gctgagtagt atttcattgt ttgtttactg cacgttttat ctagggaatg tgtgtttttt
12841 taaaaaatgg agacagctgt cctaatatga gtcaactgcc aagggctttc aattatgtct
12901 actagagttg ttaaattggc agattctaga aaatattgga ggtttacata cagtatttag
12961 acagaatagc ttcctagctt atgcaccaca ctggtgctaa ctttggcaaa gaaagcagca
13021 aagacagagt aatgttggca agcaaatcca tcgttatgca ttattaagta ttgttcatta
13081 ggctgcaaag ggtgagggaa tcacagtaat aaccactttc tgttttctgc tgcactgtat
13141 cagctcatgg aacatcttac tttgcctctg cacacagaac gaacacaatc tgtggcttgt
13201 cctgtctcaa gcctgaaggc acggcaccgc ctcagttect gatcccacag tttaaccgct
13261 cctccttctt ttgacctaag taaataacca agccagagta agtgttcatt attggctact
13321 ataattttta ttataaacaa ataccaagtt ataagcagaa tctttttttt ttaaaaaggc
13381 cctgatattt ataatttacc tctaatattc ttgtaaactt tctatggcaa tttgaggata
13441 tactatatct cagtcaaaat aaacatccag tttcagtgaa ttttattttg agaaatactc
13501 tttttttctg acatgagcat aattttattt agcctctaca atacattaca atacattatc
13561 ctctctcata atactttttt tttttttttt aagatgtagt ctcgctctgt ctcccaggct
13621 tgagtgcagt ggcatgatct aggcttattg caacctctgc ctcccaggtt caagcgattc
13681 tcctacctca gcctcccgag tagctagcat tacaggtgtg caccaccaca cccagctaat
13741 ttctgtattt ttagtagaga tggggtttca ccatgttggc caggctggtc tcaaatacct
13801 tgacctcagg tgatctgcct gcctcggcct cccaaagtgc tgggattcca ggtatgagcc
13861 actgtgcctg gcctcataat acttcttgat taggaagatg taaaaaaaca attttattaa
13921 aaggataatg gaaatgtaag gcaaaataat agaattacaa atgctatgct acagagttga
13981 tttatttatt tttttgagac agagtgtcgc tctgtcacct ggcctggagt gcagtggtgt
14041 gatctcggct cactgcaacc tgtgcctccc aggttcaagc gattcttctc cttcagcctc
14101 ccaagtagct gggattacag gcaccatgcc tggctaattt ttgtattttt agtagagatg
14161 gagtttcacc atattggcca ggctgatccc aaactectga cctcgtgate cgcccacctc
14221 ggcctcccaa agtgttggga ttacaggcgt gagccactgc aactggccca gagcttattt
14281 ttgaaggcca aaacagaagc atatttattc cctatcaggt gttaaaatat ctcactggaa
14341 cagtttagca ggcttctagt gagtgggggt gtgcaggagt aaatgacgtg ggaaatacaa
14401 gtgttggagg acgaaataga gcccatttat ggattttatt cctggaaggg ctgaaaaatg
14461 tattccttcc ttttctgcta gatgaattgc ttgtctgaaa gcatgcctat gtgcattctt
14521 cctttatgta aaaggcacaa attctgcgct tgtgtttaat taacatatgt gggttctttc
14581 aatcctgtat tgaaatgtac ttcttagtca actatatgtc acattttttt ttgtttttgt
14641 ttttgttttt taaatggggt ctcactctgt cacccaggct ggagtgcagt ggcaccatca
14701 cagctcacta aagccttgac ctccccaggc tcaagtgatc ctcccacctc agcctcctga
14761 gtagcaggga ctacaggcat gtgccaccac acccggctaa ttgttgtttt ttatagcgat
14821 ggggtttcac catgttgccc aggctggtct tgaactectg ggctcaagcg atccacctgc
14881 ctcagcctcc caaagtgata agattacagg tgtgagccac tgtgcctggc ctacatgtca
14941 tgtttcaaca tgcatatgac tatgttggtg acaaatcaaa tcataagtat ctggttactg
15001 ttgggagatt tgaaaatcac tcagaagaga cctcttctca aattttgagg tcttgtataa
15061 aacagtttaa atttgcctca agcaaaagga aacaaggcag ttctctctag ttccctcatc
15121 cttttctaaa gcaacaatgt gcattctact ccttagaatc cattctgaac aaaaagagag
15181 caggcagtca aaatacaacc ctggctccag attcccccat gggcctccta ctcagcaaat
15241 catacacagg catacagaca ttaagaaaag taactcaact tgtaggacaa ctacctatcc
15301 acacctcaga aaaagtatca ccccaacatg aaaaaaattg gaagtgaatt aagaccagaa
15361 atgagaatca aatagaaggc acataaaagg taataaagga gaagcatatg aggaggaagg
15421 tcggagagga cactctgtgt agcctagaaa caactagaat aattaactgc aaacctcagg
15481 taggtcacaa atgcataaat attctgtgaa aagaaagagg actcacggcc tttcctttcc
15541 cccagtcacg ataagtccat ctcgcagggt ggtgtacatg gcaaacacag gcccgttgtg
15601 agctctcgcc acgattctac acaatatgtg atctttccac acacagacat caccactgat
15661 ggtacctgta aacgtcaagt tattctgaaa aggagtgggg gagggggaga caaactcatc
15721 aaaagttcaa atagagttta aatagataat tttctatgta tgtgtaatgc tgtctcaccc
15781 ttgatacaaa gagcatgcat cgtgtagtgg cagcagcact gaattcacga gtcaggaaac
15841 ctgaacggga ggcttagctt tgtcaggacc ttttcctttc caagtctgtt gcttattagc
15901 tagaataacc ttagacaatt cttcccttcc aattctaaca tactataatt ctagggttta
15961 ttttttattt ttttgagacg gagtttcgct ctttgttgcc caggctggag tgcaatggtg
16021 cgatctcagc tcaccacaac ctctgcttcc caggttcaag tgattctcct gtctcagcct
16081 cccaagtagc tgggattaca agcgccagcc accacgcccg gctaattttt gtatttttag
16141 tagagacaga gtttcacctt gttagccagg ctggtcttga actcctgact tcaggtgatc
16201 ttcccgcctt ggcctcccta agtgctggga ttataggtgt gagccactgt gcccggcctg
16261 agccacggtg cctggcctgg tcttatatta agaataccca aaatgttcaa ctgaaatttg
16321 acatggcaca aacatttcaa tagtcttttt ctcaaaaatg taagtgtact taaatattct
16381 aaaattataa cttttcctat aagtattgca taatcacaaa aacaaaaaat gcacttagtt
16441 tttcgatgca ccaaaggatt tatacagcct agccaatgca ggatattaaa ggaaagagat
16501 gtggattgga agccacaggt ccagatgaga tggaataaag tgagaggaga gcaggtctcc
16561 tgaacaccct tctgtcaggg ccaggaattg tgctatttcc ttctgtctca ctacctcctt
16621 cttccctcga agtagagaca ctggcccaga gcacttccag ctgtatgata agcagtgtgt
16681 taaatgataa aaagcaaagg aaatcctaaa ccctagtacc accttaaatc atttgaaaat
16741 catgtttctt gatttacctt tctctctgac aaatttttag gactatgaag aactactagg
16801 aagacagaaa ttttaggata tttagggtga caattagaag attaaggaag gcttttgagt
16861 ataacagtag tccaaggaat caaatgttca tcagaatcct tattatggtg gctcatgcct
16921 gtaaacccag cactttggga ggtcaagatg ggaggatcac atagcttagg agcttgagac
16981 cacctaggca acatagcgaa accctgtctc tactaaaaat gaaagaaaaa ttagcctagc
17041 atggtggttc ctgccctgta gtcccagcta ctaaggaggc tgaggatcac ttgaacctgg
17101 gagatggagg ctacagtgag ctataatcgc accattgcac cccagcccag gcgacagagt
17161 gagatactgt gtcaaaaaaa aaaaaaaatc cttttccccc tctcattaac attcttttca
17221 ctccctaatt tctgaaagaa ctagattttt gaaagatgaa atatatgctt gaccagggca
17281 tgtaatgatt agcagatcac agtatcatct caacaacatt catgtggctg atgatctaag
17341 gcaagagaat gtaaagtagt caaagtcaca ctatgtgcat tttaagagac atactgcacc
17401 aaatgcaata gcgagcatgg tctgcatccg ggcatcttcc agtgtgctca gtagcccttt
17461 tttgctaaga agagctcttc ctgccagggt ccagaacttc acatgtttta ctcccactga
17521 gacaaactgg gtatctgaat ctggtcggaa ttctgccaca aaaatacgtt gattgtgacc
17581 agctctgctg gcaattttgg cacctgacaa gatacaacaa aattatctag gttattacaa
17641 gaaccaagct aatcaacagc atcaaacaaa tatgtaaaat acatagttca aaaaacaaag
17701 gcttagaaga gaggccaatg gcccctgctc tactacctag caatacatga tttacaatta
17761 tttgtgtatt gagtcctttt cacttatctt cgctccatta acttttcttt atataacgta
17821 aatgttttgt ctaaagtgtg gtaggtaata ttatcctgct gatctgccat tatcattaga
17881 aatatacata attttcataa gaatctccaa aaccaatcaa atcattaata ataaatacat
17941 agtttcttgc tggaagaaaa tagcagtgaa tcatttataa tgctaataat ggtttcatta
18001 atttatctgt tttgtgaggt tacagttcca ctgggctttt aaagtgaaat atacctacag
18061 taccactgtg tacagtatat tgcataggcc tccactgaat gattgtttca accaccaact
18121 ttaagacaaa tattaaatac agaattccta cta.
```

Disclosed herein are siRNA molecules. Also, disclosed herein are compositions comprising any of the siRNA molecules described herein or recited in Table 2. In some aspects, the siRNA molecule can be a sense strand. In some aspects, the siRNA molecule can be an antisense strand.

Disclosed herein are compositions comprising a nucleic acid sequence or molecule wherein the nucleic acid comprises or consists of a sequence having the sequence set forth in:

(SEQ ID NO: 7)
UUUUCUGGUUUCUGUGCCACACUCAGU,
(SEQ ID NO: 9)
UUUUUCUGGUUUCUGUGCCACACUCAG,
(SEQ ID NO: 11)
GUUUUUCUGGUUUCUGUGCCACACUCA,
(SEQ ID NO: 13)
AGUUUUUCUGGUUUCUGUGCCACACUC,
(SEQ ID NO: 15)
AAGUUUUUCUGGUUUCUGUGCCACACU,
(SEQ ID NO: 17)
GCAGGCCAGUACUUGCAGCGCUCCAAA,
(SEQ ID NO: 19)
AAGCAGGGAAGUAACGGCAGAGCUGAC.
(SEQ ID NO: 21)
CAAGCAGGGAAGUAACGGCAGAGCUGA,
(SEQ ID NO: 23)
ACAAGCAGGGAAGUAACGGCAGAGCUG,
(SEQ ID NO: 25)
UACAAGCAGGGAAGUAACGGCAGAGCU
(SEQ ID NO: 27)
UUACAAGCAGGGAAGUAACGGCAGAGC,
(SEQ ID NO: 29)
CUUACAAGCAGGGAAGUAACGGCAGAG,
(SEQ ID NO: 31)
UCUUACAAGCAGGGAAGUAACGGCAGA,
(SEQ ID NO: 33)
UUCUUACAAGCAGGGAAGUAACGGCAG,
(SEQ ID NO: 35)
CACUCAUCUCAGCGUUAGAAAAGCUACC,
(SEQ ID NO: 37)
UCUGGUUUCUGUGCCACACUCAGUUCAC,
(SEQ ID NO: 39)
UACUUGCAGCGCUCCAAAAGUUUUUCUG,
(SEQ ID NO: 41)
UCCCCAUUUUUACAAGCAGGCCAGUACU,
(SEQ ID NO: 43)
GAUGGGGUGAUGGUAGGCACACUCAUCC,
(SEQ ID NO: 45)
UUGGGGAAGGCUUUGCAGGGUGAGAUGG,
(SEQ ID NO: 47)
AAACAUUUUUCAGCAAAUUUACAAUUGG,
(SEQ ID NO: 49)
UAUUUACAAUUUGGGUGAACAAACAAAC,
(SEQ ID NO: 51)
UCUGGUUUAGUACACUUUGCAUCAUAUU,
(SEQ ID NO: 53)
UACUCACAUGAGUGAAGGGACAAUCUGG,
(SEQ ID NO: 55)
UUGGAGACAGUACUGGAAUUCUUCUACU,
(SEQ ID NO: 57)
GUGGUGCUGGUGGUGCAACUGGUUUUGG,
(SEQ ID NO: 59)
ACGGCAGAGCUGACUACUGGAAGGUGGU,
(SEQ ID NO: 61)
CCAUCUUCUUACAAGCAGGGAAGUAACGG,
(SEQ ID NO: 63)
GUUUUGGAUGAUAGAAGGGACAUUCCAU,
(SEQ ID NO: 65)
UACAUUGAGUGUUAAACCUACAAUGUUU,
(SEQ ID NO: 67)
GUAGAAUGUGCAGUCCGGUCUUGUACAU,
(SEQ ID NO: 69)
UGGUGGGACAUUAAUGGUGGGAUGGUAG,
(SEQ ID NO: 71)
UCGAAUCCAUUUCAAGGCAUGUCGUGGU,
or
(SEQ ID NO: 73)
UUAUUCGCUGGUUUGAGGUCGAAUCCAU.

Disclosed herein are compositions comprising a nucleic acid sequence or molecule wherein the nucleic acid comprises or consists of a sequence having at least 90% identity to the sequence set forth in:

(SEQ ID NO: 7)
UUUUCUGGUUUCUGUGCCACACUCAGU,
(SEQ ID NO: 9)
UUUUUCUGGUUUCUGUGCCACACUCAG,
(SEQ ID NO: 11)
GUUUUUCUGGUUUCUGUGCCACACUCA,
(SEQ ID NO: 13)
AGUUUUUCUGGUUUCUGUGCCACACUC,
(SEQ ID NO: 15)
AAGUUUUUCUGGUUUCUGUGCCACACU,
(SEQ ID NO: 17)
GCAGGCCAGUACUUGCAGCGCUCCAAA,
(SEQ ID NO: 19)
AAGCAGGGAAGUAACGGCAGAGCUGAC.
(SEQ ID NO: 21)
CAAGCAGGGAAGUAACGGCAGAGCUGA,
(SEQ ID NO: 23)
ACAAGCAGGGAAGUAACGGCAGAGCUG,
(SEQ ID NO: 25)
UACAAGCAGGGAAGUAACGGCAGAGCU
(SEQ ID NO: 27)
UUACAAGCAGGGAAGUAACGGCAGAGC,
(SEQ ID NO: 29)
CUUACAAGCAGGGAAGUAACGGCAGAG,
(SEQ ID NO: 31)
UCUUACAAGCAGGGAAGUAACGGCAGA,
(SEQ ID NO: 33)
UUCUUACAAGCAGGGAAGUAACGGCAG,
(SEQ ID NO: 35)
CACUCAUCUCAGCGUUAGAAAAGCUACC,
(SEQ ID NO: 37)
UCUGGUUUCUGUGCCACACUCAGUUCAC,
(SEQ ID NO: 39)
UACUUGCAGCGCUCCAAAAGUUUUUCUG,
(SEQ ID NO: 41)
UCCCCAUUUUUACAAGCAGGCCAGUACU,
(SEQ ID NO: 43)
GAUGGGGUGAUGGUAGGCACACUCAUCC,
(SEQ ID NO: 45)
UUGGGGAAGGCUUUGCAGGGUGAGAUGG,
(SEQ ID NO: 47)
AAACAUUUUUCAGCAAAUUUACAAUUGG,
(SEQ ID NO: 49)
UAUUUACAAUUUGGGUGAACAAACAAAC,
(SEQ ID NO: 51)
UCUGGUUUAGUACACUUUGCAUCAUAUU,
(SEQ ID NO: 53)
UACUCACAUGAGUGAAGGGACAAUCUGG,
(SEQ ID NO: 55)
UUGGAGACAGUACUGGAAUUCUUCUACU,
(SEQ ID NO: 57)
GUGGUGCUGGUGGUGCAACUGGUUUUGG,
(SEQ ID NO: 59)
ACGGCAGAGCUGACUACUGGAAGGUGGU,
(SEQ ID NO: 61)
CCAUCUUCUUACAAGCAGGGAAGUAACGG,
(SEQ ID NO: 63)
GUUUUGGAUGAUAGAAGGGACAUUCCAU,
(SEQ ID NO: 65)
UACAUUGAGUGUUAAACCUACAAUGUUU,
(SEQ ID NO: 67)
GUAGAAUGUGCAGUCCGGUCUUGUACAU,
(SEQ ID NO: 69)
UGGUGGGACAUUAAUGGUGGGAUGGUAG,
(SEQ ID NO: 71)
UCGAAUCCAUUUCAAGGCAUGUCGUGGU,
or
(SEQ ID NO: 73)
UUAUUCGCUGGUUUGAGGUCGAAUCCAU.

TABLE 2
Examples of siRNA Sequences

| | | SEQ | | SEQ |
|---|---|---|---|---|
| | | ID | | ID |
| Name | Sense | NO: | Anti-Sense | NO: |
| MnH MSU | UGAGUGUGGCACAG | 6 | UUUUCUGGUUUCUGU | 7 |
| U2si4 | AAACCAGAAAA | | GCCACACUCAGU | |
| MnH_MSU | GAGUGUGGCACAGA | 8 | UUUUUCUGGUUUCUG | 9 |
| U2si5 | AACCAGAAAAA | | UGCCACACUCAG | |
| MnH_MSU | AGUGUGGCACAGAA | 10 | GUUUUUCUGGUUUCU | 11 |
| U2si6 | ACCAGAAAAAC | | GUGCCACACUCA | |
| MnH_MSU | GUGUGGCACAGAAA | 12 | AGUUUUUCUGGUUUC | 13 |
| U2si7 | CCAGAAAAACU | | UGUGCCACACUC | |
| MnH_MSU | UGUGGCACAGAAAC | 14 | AAGUUUUUCUGGUU | 15 |
| U2si8 | CAGAAAAACUU | | UCUGUGCCACACU | |
| MnH_MSU | UGGAGCGCUGCAAG | 16 | GCAGGCCAGUACUUG | 17 |
| U2si9 | UACUGGCCUGC | | CAGCGCUCCAAA | |
| MnH_MSU | CAGCUCUGCCGUUAC | 18 | AAGCAGGGAAGUAAC | 19 |
| U2si10 | UUCCCUGCUU | | GGCAGAGCUGAC | |
| MnH_MSU | AGCUCUGCCGUUAC | 20 | CAAGCAGGGAAGUAA | 21 |
| U2sill | UUCCCUGCUUG | | CGGCAGAGCUGA | |
| MnH_MSU | GCUCUGCCGUUACU | 22 | ACAAGCAGGGAAGUA | 23 |
| U2si12 | UCCCUGCUUGU | | ACGGCAGAGCUG | |
| MnH_MSU | CUCUGCCGUUACUUC | 24 | UACAAGCAGGGAAGU | 25 |
| U2si13 | CCUGCUUGUA | | AACGGCAGAGCU | |
| MnH_MSU | UCUGCCGUUACUUCC | 26 | UUACAAGCAGGGAAG | 27 |
| U2si14 | CUGCUUGUAA | | UAACGGCAGAGC | |
| MnH_MSU | CUGCCGUUACUUCCC | 28 | CUUACAAGCAGGGAA | 29 |
| U2si15 | UGCUUGUAAG | | GUAACGGCAGAG | |
| MnH_MSU | UGCCGUUACUUCCCU | 30 | UCUUACAAGCAGGGA | 31 |
| U2si16 | GCUUGUAAGA | | AGUAACGGCAGA | |
| MnH_MSU | GCCGUUACUUCCCUG | 32 | UUCUUACAAGCAGGG | 33 |
| U2si17 | CUUGUAAGAA | | AAGUAACGGCAG | |
| hMSsiwalk | GGUAGCUUUUCUAA | 34 | CACUCAUCUCAGCGU | 35 |
| 28 | CGCUGAGAUGAGUG | | UAGAAAAGCUACC | |
| hMSsiwalk | GUGAACUGAGUGUG | 36 | UCUGGUUUCUGUGCC | 37 |
| 53 | GCACAGAAACCAGA | | ACACUCAGUUCAC | |
| hMSsiwalk | CAGAAAAACUUUUG | 38 | UACUUGCAGCGCUCC | 39 |
| 77 | GAGCGCUGCAAGUA | | AAAAGUUUUUCUG | |
| hMSsiwalk | AGUACUGGCCUGCU | 40 | UCCCCAUUUUUACAA | 41 |
| 101 | UGUAAAAAUGGGGA | | GCAGGCCAGUACU | |
| hMSsiwalk | GGAUGAGUGUGCCU | 42 | GAUGGGGUGAUGGU | 43 |
| 126 | ACCAUCACCCCAUC | | AGGCACACUCAUCC | |
| hMSsiwalk | CCAUCUCACCCUGCA | 44 | UUGGGGAAGGCUUU | 45 |
| 149 | AAGCCUUCCCCAA | | GCAGGGUGAGAUGG | |
| hMSsiwalk | CCAAUUGUAAAUUU | 46 | AAACAUUUUUCAGCA | 47 |
| 173 | GCUGAAAAAUGUUU | | AAUUUACAAUUGG | |
| hMSsiwalk | GUUUGUUUGUUCAC | 48 | UAUUUACAAUUUGG | 49 |
| 197 | CCAAAUUGUAAAUA | | GUGAACAAACAAAC | |
| hMSsiwalk | AAUAUGAUGCAAAG | 50 | UCUGGUUUAGUACAC | 51 |
| 221 | UGUACUAAACCAGA | | UUUGCAUCAUAUU | |
| hMSsiwalk | CCAGAUUGUCCCUUC | 52 | UACUCACAUGAGUGA | 53 |
| 244 | ACUCAUGUGAGUA | | AGGGACAAUCUGG | |
| hMSsiwalk | AGUAGAAGAAUUCC | 54 | UUGGAGACAGUACUG | 55 |
| 268 | AGUACUGUCUCCAA | | GAAUUCUUCUACU | |
| hMSsiwalk | CCAAAACCAGUUGC | 56 | GUGGUGCUGGUGGU | 57 |
| 292 | ACCACCAGCACCAC | | GCAACUGGUUUUGG | |
| hMSsiwalk | ACCACCUUCCAGUAG | 58 | ACGGCAGAGCUGACU | 59 |
| 315 | UCAGCUCUGCCGU | | ACUGGAAGGUGGU | |
| hMSsiwalk | CCGUUACUUCCCUGC | 60 | CCAUCUUCUUACAAG | 61 |
| 340 | UUGUAAGAAGAUGG | | CAGGGAAGUAACGG | |
| hMSsiwalk | AUGGAAUGUCCCUU | 62 | GUUUUGGAUGAUAG | 63 |
| 364 | CUAUCAUCCAAAAC | | AAGGGACAUUCCAU | |
| hMSsiwalk | AAACAUUGUAGGUU | 64 | UACAUUGAGUGUUA | 65 |
| 388 | UAACACUCAAUGUA | | AACCUACAAUGUUU | |
| hMSsiwalk | AUGUACAAGACCGG | 66 | GUAGAAUGUGCAGUC | 67 |
| 411 | ACUGCACAUUCUAC | | CGGUCUUGUACAU | |
| hMSsiwalk | CUACCAUCCCACCAU | 68 | UGGUGGGACAUUAA | 69 |
| 408 | UAAUGUCCCACCA | | UGGUGGGAUGGUAG | |
| hMSsiwalk | ACCACGACAUGCCUU | 70 | UCGAAUCCAUUUCAA | 71 |
| 432 | GAAAUGGAUUCGA | | GGCAUGUCGUGGU | |
| hMSsiwalk | AUGGAUUCGACCUC | 72 | UUAUUCGCUGGUUUG | 73 |
| 450 | AAACCAGCGAAUAA | | AGGUCGAAUCCAU | |

In some aspects, a siRNA molecule can comprise a double-stranded RNA molecule. In some aspects, the siRNA molecule can comprise a double-stranded RNA molecule whose antisense strand will comprise an RNA sequence substantially complementary to at least one sequence consisting of SEQ ID NO: 1 to SEQ ID NO: 5, SEQ ID NO: 77 or SEQ ID NO: 78, and whose sense strand will comprise an RNA sequence complementary to the antisense strand, wherein both strands are hybridised by standard base pairing between nucleotides. In some aspects, a siRNA molecule can comprise a double stranded RNA molecule, whose antisense strand will comprise an RNA sequence substantially complementary to SEQ ID NO: 1 to SEQ ID NO: 5, SEQ ID NO: 77 or SEQ ID NO: 78.

As used herein, “substantially complementary” to a target mRNA sequence, can also be understood as “substantially identical” to said target sequence. “Identity” is the degree of sequence relatedness between nucleotide sequences as determined by matching the order and identity of nucleotides between sequences. In some aspects, the antisense strand of an siRNA having 85%, 86%, 87%, 88%, 89%, 90%, 91%, 92%, 93%, 94%, 95%, 96%, 97%, 98% or 99% complementarity to the target mRNA sequence are considered substantially complementary and may be used in the present invention. The percentage of complementarity describes the percentage of contiguous nucleotides in a first nucleic acid molecule that can base pair in the Watson-Crick sense with a set of contiguous nucleotides in a second nucleic acid molecule. In some aspects, the antisense siRNA strand is 100% complementary to the target mRNA sequence, and the sense strand is 100% complementary to the antisense strand over the double stranded portion of the siRNA. The siRNA may also include unpaired overhangs, for example, 3′ dinucleotide overhangs, and, in some aspects, dTdT.

Generally, double stranded molecules can be from about 19 to about 25 nucleotides in length, and include blunt-ended structures as well as those with overhangs. Overhangs have been described to be advantageous and may be present on the 5′ ends or on the 3′ ends of either strand as they reduce recognition by RNAses and imitate Dicer's natural substrate. In some aspects, overhangs can be present on both 3′ ends of the molecules. In some aspects one overhang is present on one end of the molecule. Others have described the use of blunt-ended structures with specific modification patterns (EP1527176, WO2005062937, WO2008104978, EP2322617, EP2348133, US20130130377, and many others).

Overhangs can comprise between 1 and 5 nucleotides; typically overhangs are made up of dinucleotides. Classical molecules used in the field, comprise a 19 nucleotide double stranded molecule which further comprises 3′ dinucleotide overhangs preferably comprising deoxynucleotides as taught in initial studies by Tuschl (WO0244321). These overhangs are said to further enhance resistance to nuclease (RNase) degradation. Later, Kim et al. 2005 (Kim et al., Nat. Biotechnol. 2005, February; 23(2): 222-6) describe that 21-mer products (containing dinucleotide overhangs) are important for loading onto RNA-induced silencing complex (RISC). Further, Bramsen et al. 2009 (Bramsen et al. Nucleic Acids Res. 2009, May; 37(9): 2867-81) describe the introduction of possible destabilizing modifications to the overhangs to further increase silencing efficiency.

In some aspects, the siRNA molecules described herein can target at least one sequence selected from the group consisting of SEQ ID NO: 1 to SEQ ID NO: 5, SEQ ID NO: 77 and SEQ ID NO: 78 which comprises at least one overhang, preferably a 3′ overhang in the sense and/or the antisense strand. In some aspects, wherein the siRNA molecule targets at least one sequence selected from the group consisting of SEQ ID NO: 1 to SEQ ID NO: 5, SEQ ID NO: 77 or SEQ ID NO: 78, the siRNA can include an antisense strand of equivalent length and complementary to the target, and a sense strand of equivalent length and complementary to the antisense strand. The antisense and sense strands can further include additional bases which are not complementary to the other strand or the target, and/or which are not paired in the double stranded portion of the siRNA.

In some aspects, the siRNA molecules described herein that target at least one sequence selected from the group consisting of SEQ ID NO: 1 to SEQ ID NO: 5, SEQ ID NO: 77 and SEQ ID NO: 78, wherein each strand of the double-stranded siRNA molecules is about 18 to about 28 or more (e.g., about 18, 19, 20, 21, 22, 23, 24, 25, 26, 27, or 28 or more) nucleotides long.

Disclosed herein are siRNA molecules wherein the siRNA molecule specifically targets a sequence comprising or consisting of a sequence having the sequence of SEQ ID NO: 1, 2, 3, 4, 5, 77 or 78 and reduces expression of mammalian suppressor of tauopathy 2 (MSUT2) gene in a cell, wherein the siRNA molecule comprises a 25- to 28-nucleotide blunt-ended double-stranded structure, wherein the siRNA molecule comprises at least one sequence having at least 90% sequence identity to a sequence comprising the sequence of SEQ ID NO: 6 to SEQ ID NO: 73

In some aspects, the siRNA molecules described herein comprising 18-28 nucleotides long or more and comprising a nucleotide sequence selected from the group consisting of SEQ ID NO: 6 to SEQ ID NO: 73. In some aspects, the siRNA molecules described herein comprising 18-28 nucleotides long or more and comprising a nucleotide sequence selected from the group consisting of SEQ ID NOs: 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, and 73. In some aspects, the double-stranded siRNA molecules can be at least 19 nucleotides long and selected from the group consisting of SEQ ID NO: 6 to SEQ ID NO: 73.

Also described herein are blunt-ended molecules. Disclosed herein are siRNA molecules wherein the siRNA molecules specifically target at least one sequence selected from the group consisting of SEQ ID NO: 1 to SEQ ID NO: 5, SEQ ID NO: 77 and SEQ ID NO: 78. In some aspects, the siRNA molecules can reduce expression of mammalian suppressor of tauopathy 2 (MSUT2) gene in a cell. In some aspects, the siRNA molecules comprise an 18- to 28-nucleotide, a 19- to 25-nucleotide or a 25- to 28-nucleotide blunt-ended double-stranded structure. In some aspects, the siRNA molecule comprises at least one sequence having at least 90% a sequence identity selected from the group consisting of SEQ ID NO: 6 to SEQ ID NO: 73. In some aspects, the siRNA molecule comprises at least one sequence having at least 90% a sequence identity selected from the group consisting of SEQ ID NOs: 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, and 73.

In some aspects, the siRNA molecules comprise a 19 nucleotide double-stranded blunt-ended siRNA targeted against at least one sequence selected from the group consisting of SEQ ID NO: 1 to SEQ ID NO: 5, SEQ ID NO: 77 and SEQ ID NO: 78. In some aspects, the siRNA molecule comprises or consists of at least one sequence selected from the group consisting of SEQ ID NO: 6 to SEQ ID NO: 73. In some aspects, the antisense strand of this siRNA is at least 80%, at least 90%, complementary to at least one sequence selected from the group consisting of SEQ ID NO: 1 to SEQ ID NO: 5, SEQ ID NO: 77 and SEQ ID NO: 78.

In some aspects, the siRNA molecules disclosed herein can comprise or consist of at least one sequence selected from the group consisting of SEQ ID NO: 6 to SEQ ID NO: 73. In some aspects, the siRNA molecules disclosed herein can comprise or consist of at least one sequence selected from the group consisting of SEQ ID NOs: 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, and 73.

In some aspects, the siRNA molecules disclosed herein can comprise or consist a sense strand which comprises or consists of at least one sequence selected from the group consisting of SEQ ID NOs: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72, and an antisense strand which is complementary to the sense strand.

siRNA molecules can be unstable in biological fluids due to the ubiquitous nature of RNAses. Thus, the use of many different chemical modifications to nucleotides has been described with the purpose of enhancing compound stability. Disclosed herein are siRNA molecules that are stability in biological fluids.

siRNA molecules can be immunogenetic, and in some instance, have been found to induce unspecific activation of the innate immune system, including up-regulation of certain cytokines.

Both of these effects, recognition by RNases and immunogenicity, have also been described to be sequence-dependent.

Described herein are chemical modifications that can enhance or are capable of enhancing siRNA molecule stability. In some aspects, the chemical modification can increase or enhance siRNA molecule stability by decreasing its susceptibility to RNAses as well as reduce induction of immune recognition and thus reduce the subsequent immune response.

In some aspects, the siRNA molecules described herein can further comprise at least one nucleotide with a chemical modification. In some aspects, at least one nucleotide of the siRNA molecule can comprise a chemical modification.

In some aspects, the chemical modification(s) that enhances stability and reduces immunogenic effects can include but is not limited to 2′-O-methyl nucleotides, 2′-fluoro nucleotides, 2′-amino nucleotides, 2′-deoxy nucleotides, or nucleotides containing 2′-O or 4′-C methylene bridges. Examples of chemical modifications for exonuclease protection include but are not limited to the ExoEndoLight pattern of modification (EEL): modification of the pyrimidines in the sense strand to 2′-O-methyl residues, and modification of the pyrimidines in a 5′-UA-3′ or 5′-CA-3′ motif in the antisense strand to 2′-O-methyl residues. In some aspects, position 1 of the sense strand can also be changed to 2′-O-methyl to prevent 5′-phosphorylation of the sense strand and thus increasing strand-specificity of the siRNA. In addition, the sense strand can also include a 2′-O-methyl modification in position 14, because 2′-O-Me residues at this position inactivate the sense strand and therefore increase strand-specificity of the siRNA molecules. Additional examples of chemical modifications for nuclease protection include but are not limited to Methyl-Fluoro modification pattern (MEF): alternating 2′-fluoro and 2′-O-methyl modifications starting (5′-end) with a 2′-F on the sense strand and starting with 2′-O-Me on the antisense strand. In some aspects, position 1 of the sense strand can also be changed to 2′-O-Me and position 1 of the antisense strand to 2′-F (as 2′F residues are compatible with 5′-phosphorylation whereas 2′O-Me residues are bulky and generally impair phosphorylation). This modification pattern can stabilize the molecule as well as disable the ability of the RISC to use the sense strand thus promoting strand-specificity. Also, modification of the ribonucleotide backbone can be performed by binding the nucleotides by using phosphorothioate bonds instead of phosphodiester links. In some aspects, the chemical modification can be a 4′Thioribose, 5-Propynyluracil 3′,5′-methyluridine or the substitution of uracyl ribonucleotides with deoxythymidine (deoxyribonucleotides).

In some aspects, the chemical modification can include one or more amino acids, with amino acid, carbohydrates, or lipid moieties.

In some aspects, the at least one chemically modified nucleotide and/or the at least one chemical modification in the ribonucleotide backbone is on the sense strand, on the antisense strand or on both strands of the siRNA molecule. In some aspects, the chemical modification is on the sense strand, on the antisense strand or on both strands of the siRNA molecule.

In some aspects, the siRNA molecule can comprise or consist of at least one sequence with a sense strand and/or an antisense strand selected from the group consisting of SEQ ID NO: 6 to SEQ ID NO: 73. In some aspects, the siRNA molecule can comprise or consist of at least one sequence selected from the group consisting of SEQ ID NO: 6 to SEQ ID NO: 73.

In some aspects, the siRNA molecule can comprise or consists of a sense strand which comprises or consists of at least one sequence selected from the group of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64 SEQ ID NO: 66, SEQ ID NO: 68, SEQ ID NO: 70, and SEQ ID NO: 72.

In some aspects, the siRNA molecule can comprise or consists of an antisense strand which is complementary to the sense strand which is selected from the group of SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65 SEQ ID NO: 67, SEQ ID NO: 69, SEQ ID NO: 71, and SEQ ID NO: 73.

In some aspects, the siRNA molecule can comprise or consist of a sense strand which comprises or consists of at least one sequence selected from the group of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64 SEQ ID NO: 66, SEQ ID NO: 68, SEQ ID NO: 70, and SEQ ID NO: 72; and an antisense strand which is complementary to the sense strand which is selected from the group of SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65 SEQ ID NO: 67, SEQ ID NO: 69, SEQ ID NO: 71, and SEQ ID NO: 73.

Any of the compositions disclosed herein can further comprise a pharmaceutically acceptable carrier. In some aspects, the pharmaceutically acceptable carrier for the siRNA molecule can be buffered saline. In some aspects, the pharmaceutically acceptable carrier can comprise a lipid-based or polymer-based colloid. In some aspects, the colloid can be a liposome, a hydrogel, a microparticle, a nanoparticle, or a block copolymer micelle. In some aspects, the compositions described herein can be formulated for intravenous, subcutaneous, intrathecal, intramuscular, oral, intrathecal or intraperitoneal administration. In some aspects, the therapeutically effective amount of any of the siRNA molecules disclosed herein reduces accumulation of phosphorylated and aggregated human tau.

siRNA molecules described herein can be delivered to the cell interior in their native structure using methods known in the art. In some aspects, when the siRNA molecules can be administered using standard transfection reagents. To achieve effects in vivo these siRNA molecules can also be administered naked or using delivery enhancing agents such as for example liposomes, conjugation with a specific moiety, etc. although many different alternatives are known in the art, and are used differently depending on the desired target site within the body.

In some aspects, the siRNA molecules described herein can be expressed within cells from eukaryotic promoters. Recombinant vectors capable of expressing the siRNA molecules can be delivered and persist in target cells. Alternatively, vectors can be used that provide for transient expression of nucleic acid molecules. Such vectors can be repeatedly administered as necessary. Once expressed, the siRNA molecule interacts with the target mRNA and generates an RNA interfering response. The siRNA molecules produced in this manner are often termed shRNA (short hairpin RNA), as their sense and antisense strands are joined by a small loop of nucleotides. Delivery of siRNA molecules expressing vectors can be systemic, such as by intravenous or intra-muscular administration, by administration to target cells ex-planted from a subject followed by reintroduction into the subject, or by any other means that would allow for introduction into the desired target cell.

Also disclosed is the use of siRNA targeting at least one sequence selected from the group consisting of SEQ ID NO: 1 to SEQ ID NO: 5, SEQ ID NO: 77 and SEQ ID NO: 78 in the preparation of a medicament for use in a method of treatment Alzheimer's disease or dementia characterized by increased expression and/or activity of MSUT2. In some aspects, the use comprises inhibiting expression of MSUT2 polynucleotide in a subject. The term inhibition is used to indicate a decrease or downregulation of expression or activity. In some aspects, the Alzheimer's disease or dementia can be associated with or related to an increase in phosphorylated or aggregated tau protein.

### Method of Treatment

The methods disclosed herein can be useful for the treatment of a subject with Alzheimer's disease or dementia. In some aspects, the siRNA molecule can potentiate the neuroinflammatory response to pathological tau. In some aspects, the siRNA molecule can decrease astrocytosis and microgliosis. In some aspects, the siRNA molecule can reduce neuroinflammation. In some aspects, the siRNA molecule can inhibit expression of a MUST2 polynucleotide. In some aspects, the siRNA molecule can reduce accumulation of phosphorylated and aggregated human tau.

In some aspects, the methods can comprise administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the therapeutically effective amount can reduce accumulation of phosphorylated and aggregated human tau.

In some aspects, the methods can comprise administering to a subject with Alzheimer's disease or dementia a therapeutically effective amount of a small interfering RNA (siRNA) molecule or a composition comprising a siRNA molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the therapeutically effective amount can reduce accumulation of phosphorylated and aggregated human tau.

The methods disclosed herein can be useful for inhibiting expression of a MSUT2 polynucleotide. In some aspects, the method can inhibit expression of a MSUT2 polynucleotide in a subject. The method can comprise administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73, SEQ ID NO: 77 and SEQ ID NO: 78. In some aspects, the therapeutically effective amount can reduce accumulation of phosphorylated and aggregated human tau.

The methods disclosed herein can be useful for inhibiting expression of a MSUT2 polynucleotide. In some aspects, the method can inhibit expression of a MSUT2 polynucleotide in a subject. The method can comprise administering to a subject with Alzheimer's disease or dementia a therapeutically effective amount of a small interfering RNA (siRNA) molecule or a composition comprising a siRNA molecule wherein the siRNA molecule comprises a 25- to 28-nucleotide blunt-ended double-stranded structure, wherein the siRNA molecule comprises at least one sequence of SEQ ID NO: 6 to SEQ ID NO: 73. In some aspects, the siRNA molecule can comprise at least one sequence having at least 90% sequence identity to a sequence of SEQ ID NO: 6 to SEQ ID NO: 73, SEQ ID NO: 77 and SEQ ID NO: 78. In some aspects, the therapeutically effective amount can reduce accumulation of phosphorylated and aggregated human tau.

The methods disclosed herein can be useful for reducing phosphorylated and aggregated human tau protein in a subject. The methods can comprise administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the therapeutically effective amount can reduce accumulation of phosphorylated and aggregated human tau.

The methods disclosed herein can be useful for reducing phosphorylated and aggregated human tau protein in a subject. The methods can comprise administering to a subject with Alzheimer's disease or dementia a therapeutically effective amount of a small interfering RNA (siRNA) molecule or a composition comprising a siRNA molecule wherein the siRNA molecule comprises a 25- to 28-nucleotide blunt-ended double-stranded structure, wherein the siRNA molecule comprises at least one sequence of SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the therapeutically effective amount can reduce accumulation of phosphorylated and aggregated human tau.

The methods disclosed herein can be useful for suppressing expression of a MSUT2 polynucleotide. In some aspects, the method can suppress expression of a MSUT2 polynucleotide in a subject. The method can comprise administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the therapeutically effective amount can reduce accumulation of phosphorylated and aggregated human tau.

The methods disclosed herein can be useful for suppressing expression of a MSUT2 polynucleotide. In some aspects, the method can suppress expression of a MSUT2 polynucleotide in a subject. The method can comprise administering to a subject with Alzheimer's disease or dementia a therapeutically effective amount of a small interfering RNA (siRNA) molecule or a composition comprising a siRNA molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the therapeutically effective amount can reduce accumulation of phosphorylated and aggregated human tau.

The methods disclosed herein can be useful for potentiating a neuroinflammatory response to a pathological tau protein. In some aspects, the method can potentiate a neuroinflammatory response to a pathological tau protein in a subject. The method can comprise administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the therapeutically effective amount can reduce accumulation of phosphorylated and aggregated human tau.

The methods disclosed herein can be useful for potentiating a neuroinflammatory response to a pathological tau protein. In some aspects, the method can potentiate a neuroinflammatory response to a pathological tau protein in a subject. The method can comprise administering to a subject with Alzheimer's disease or dementia a therapeutically effective amount of a small interfering RNA (siRNA) molecule or a composition comprising a siRNA molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the therapeutically effective amount can reduce accumulation of phosphorylated and aggregated human tau.

The methods disclosed herein can be useful for decreasing astrocytosis or microgliosis. In some aspects, the method can decrease astrocytosis or microgliosis in a subject. The method can comprise administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the therapeutically effective amount can reduce accumulation of phosphorylated and aggregated human tau.

The methods disclosed herein can be useful for decreasing astrocytosis or microgliosis. In some aspects, the method can decrease astrocytosis or microgliosis in a subject. The method can comprise administering to a subject with Alzheimer's disease or dementia a therapeutically effective amount of a small interfering RNA (siRNA) molecule or a composition comprising a siRNA molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the therapeutically effective amount can reduce accumulation of phosphorylated and aggregated human tau.

The methods disclosed herein can be useful for reducing neuroinflammation. In some aspects, the method can reduce neuroinflammation in a subject. The method can comprise administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the therapeutically effective amount can reduce accumulation of phosphorylated and aggregated human tau.

The methods disclosed herein can be useful for reducing neuroinflammation. In some aspects, the method can reduce neuroinflammation in a subject. The method can comprise administering to a subject with Alzheimer's disease or dementia a therapeutically effective amount of a small interfering RNA (siRNA) molecule or a composition comprising a siRNA molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the therapeutically effective amount can reduce accumulation of phosphorylated and aggregated human tau.

In some aspects, the subject has Alzheimer's disease. In some aspects, the subject has dementia. In some aspects, the subject has mild-moderate Alzheimer's disease. In some aspects, the subject has moderate-severe Alzheimer's disease. Alzheimer's disease typically progresses slowly in three general stages, mild (early stage), moderate (middle stage) and severe (late stage). In mild Alzheimer's disease (early stage), subjects can still function independently but may notice that they are having memory lapses such as forgetting familiar words or the location of everyday objects. During moderate Alzheimer's disease (middle stage), subjects may have greater difficulty performing tasks (e.g., paying bills) and confusing words, but may still remember significant details about their life. In addition, subjects in this stage may feel moody or withdrawn, are at an increased risk of wandering and becoming lost, and can exhibit personality and behavioral changes including suspiciousness and delusions or compulsive, repetitive behavior. In severe Alzheimer's disease (late stage), subjects lose the ability to respond to their environment, to carry on a conversation and eventually, to control movement. Also, during this severe stage, subjects need extensive help with daily activities and have increasing difficulty communicating.

In some aspects, the subject has an Alzheimer's-related dementia. In some aspects, the Alzheimer's-related dementia can be progressive supranuclear palsy, chronic traumatic encephalopathy, frontotemporal lobar degeneration, or other tauopathy disorders. The methods disclosed herein can be effective for targeting one or more genes, including mammalian suppressor of tauopathy 2 (MSUT2).

In some aspects, the methods also include the step of administering a therapeutic effective amount of any of the siRNA molecules disclosed herein. In some aspects, siRNA molecule comprises or consists of a sense strand which comprises or consists of at least one sequence selected from the group of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64 SEQ ID NO: 66, SEQ ID NO: 68, SEQ ID NO: 70, and SEQ ID NO: 72.

In some aspects, siRNA molecule comprises or consists of an anti-sense strand which comprises or consists of at least one sequence selected from the group of SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65 SEQ ID NO: 67, SEQ ID NO: 69, SEQ ID NO: 71, and SEQ ID NO: 73.

In some aspects, the siRNA molecule comprises or consists of a sense strand which comprises or consists of at least one sequence selected from the group of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64 SEQ ID NO: 66, SEQ ID NO: 68, SEQ ID NO: 70, and SEQ ID NO: 72; and an antisense strand which is complementary to the sense strand which is selected from the group of SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65 SEQ ID NO: 67, SEQ ID NO: 69, SEQ ID NO: 71, and SEQ ID NO: 73.

In some aspects, the methods of treating a subject can comprise contacting a cell or a subject with an effective amount of a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the siRNA molecule reduces accumulation of phosphorylated and aggregated tau.

In some aspects, the methods of treating a subject can comprise contacting a cell or a subject with an effective amount of a small interfering RNA (siRNA) molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the siRNA molecule reduces accumulation of phosphorylated and aggregated tau.

Disclosed herein are methods of inhibiting expression of a MSUT2 polynucleotide. In some aspects, the methods can comprise contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the siRNA molecule reduces accumulation of phosphorylated and aggregated tau.

In some aspects, the methods can comprise contacting a cell with a small interfering RNA (siRNA) molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the siRNA molecule reduces accumulation of phosphorylated and aggregated tau.

Disclosed herein are methods of suppressing expression of a MSUT2 polynucleotide. In some aspects, the methods can comprise contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the siRNA molecule reduces accumulation of phosphorylated and aggregated tau.

In some aspects, the methods can comprise contacting a cell with a small interfering RNA (siRNA) molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the siRNA molecule reduces accumulation of phosphorylated and aggregated tau.

Disclosed herein are methods of potentiating a neuroinflammatory response to a pathological tau protein. In some aspects, the methods can comprise contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the siRNA molecule reduces accumulation of phosphorylated and aggregated tau.

In some aspects, the methods can comprise contacting a cell with a small interfering RNA (siRNA) molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the siRNA molecule reduces accumulation of phosphorylated and aggregated tau.

Disclosed herein are methods decreasing astrocytosis or microgliosis. In some aspects, the methods can comprise contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the siRNA molecule reduces accumulation of phosphorylated and aggregated tau.

In some aspects, the methods can comprise contacting a cell with a small interfering RNA (siRNA) molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the siRNA molecule reduces accumulation of phosphorylated and aggregated tau.

Disclosed herein are methods reducing neuroinflammation. In some aspects, the methods can comprise contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78. In some aspects, the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73. In some aspects, the siRNA molecule reduces accumulation of phosphorylated and aggregated tau.

In some aspects, the methods can comprise contacting a cell with a small interfering RNA (siRNA) molecule wherein the siRNA molecule comprises a 25- to 28-nucleotide blunt-ended double-stranded structure, wherein the siRNA molecule comprises at least one sequence selected from SEQ ID NO: 6 to SEQ ID NO: 73. In some aspects, the siRNA molecule can comprise at least one sequence having at least 90% sequence identity to a sequence selected from SEQ ID NO: 6 to SEQ ID NO: 73. In some aspects, the siRNA molecule reduces accumulation of phosphorylated and aggregated tau.

In some aspects, the cell can be a vertebrate, a mammalian or a human cell. In some aspects, the cell can be a brain cell. In some aspects, the cell can be a mammalian cell. In some aspects, the mammalian cell can be a brain cell.

In some aspects, at least one nucleotide of any of the siRNA molecules can comprise a chemical modification. In some aspects, the chemical modification can be on the sense strand, the antisense strand or on both. In some aspects, the siRNA molecule can comprise at least one sequence is selected from the group consisting of SEQ ID NO: 6-SEQ ID NO: 73. In some aspects, the siRNA molecule can comprise at least one sequence having at least 90% sequence identity to a sequence selected from the group consisting of SEQ ID NO: 6 to SEQ ID NO: 73.

In some aspects, the methods can further include the step of identifying a subject (e.g., a human patient) who has Alzheimer's disease or dementia and then providing to the subject any of the siRNA molecules disclosed herein or a composition comprising any of the siRNA molecules disclosed herein. In some aspects, the small interfering RNA (siRNA) molecule or the composition comprising the siRNA molecule specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73. In some aspects, the siRNA molecule can comprise at least one sequence having at least 90% sequence identity to a sequence selected from the group consisting of SEQ ID NO: 6 to SEQ ID NO: 73.

In some aspects, the subject has an Alzheimer's-related dementia. In some aspects, the Alzheimer's-related dementia can be progressive supranuclear palsy, chronic traumatic encephalopathy, frontotemporal lobar degeneration, or other tauopathy disorders. In some aspects, the subject can be identified using standard clinical tests known to those skilled in the art. While a definite AD diagnosis requires post-mortem examination, skilled clinicians can conduct an evaluation of cognitive function with over 95% accuracy. Examples of tests for diagnosing Alzheimer's disease or dementia include Mini-Mental State Examination (MMSE), Mini-Cog© Score, Alzheimer's Disease Composite Score (ADCOMS), Alzheimer's Disease Assessment Scale-cognitive subscale (ADAS-Cog) and Clinical Dementia Rating Sum of Boxes (CDR-SB).

The therapeutically effective amount can be the amount of the composition administered to a subject that leads to a full resolution of the symptoms of the condition or disease, a reduction in the severity of the symptoms of the condition or disease, or a slowing of the progression of symptoms of the condition or disease. The methods described herein can also include a monitoring step to optimize dosing. The compositions described herein can be administered as a preventive treatment or to delay or slow the progression of degenerative changes. In some aspects, the therapeutically effective amount of any of the siRNA molecules disclosed herein can reduce accumulation of phosphorylated and aggregated human tau.

The compositions disclosed herein can be used in a variety of ways. For instance, the compositions disclosed herein can be used for direct delivery of modified therapeutic cells, or adeno-associated virus. The compositions disclosed herein can be used or delivered or administered at any time during the treatment process. The compositions described herein including cells or a virus can be delivered to the one or more brain regions, one or more brain cells, or to brain regions or brain cells to stop or prevent one or more signs of symptoms of the disease or condition in an adjacent brain region or brain cell.

The dosage to be administered depends on many factors including, for example, the route of administration, the formulation, the severity of the patient's condition/disease, previous treatments, the patient's size, weight, surface area, age, and gender, other drugs being administered, and the overall general health of the patient including the presence or absence of other diseases, disorders or illnesses. Dosage levels can be adjusted using standard empirical methods for optimization known by one skilled in the art. Administrations of the compositions described herein can be single or multiple (e.g., 2- or 3-, 4-, 6-, 8-, 10-, 20-, 50-, 100-, 150-, or more fold). Further, encapsulation of the compositions in a suitable delivery vehicle (e.g., polymeric microparticles or implantable devices) can improve the efficiency of delivery.

The therapeutically effective amount of the compositions described herein can include a single treatment or a series of treatments (i.e., multiple treatments or administered multiple times). Treatment duration using any of compositions disclosed herein can be any length of time, such as, for example, one day to as long as the life span of the subject (e.g., many years). For instance, the composition can be administered daily, weekly, monthly, yearly for a period of 5 years, ten years, or longer. The frequency of treatment can vary. For example, the compositions described herein can be administered once (or twice, three times, etc.) daily, weekly, monthly, or yearly for a period of 5 years, ten years, or longer.

In some aspects, the compositions disclosed herein can also be co-administered with another therapeutic agent. In some aspects, the methods disclosed herein can further comprise administering a cholinesterase inhibitor to the subject. In some aspects, the cholinesterase inhibitor can be galantamine, rivastigmine or donepezil. In some aspects, the methods disclosed herein can further comprise administering an anti-inflammatory therapy to the subject.

In some aspects, the methods disclosed herein also include treating a subject having Alzheimer's disease or dementia. In some aspects, the methods disclosed herein can include the step of determining MSUT2 levels in a subject.

### Pharmaceutical Compositions

As disclosed herein, are pharmaceutical compositions, comprising the compositions disclosed herein. In some aspects, the pharmaceutical composition can comprise any of siRNA molecules disclosed herein. In some aspects, the compositions can comprise at least one siRNA molecule disclosed herein. In some aspects, the pharmaceutical compositions can further comprise a pharmaceutically acceptable carrier.

Disclosed herein, are pharmaceutical compositions, comprising a nucleic acid sequence or molecule wherein the nucleic acid comprises or consists of a sequence having the sequence set forth in:

(SEQ ID NO: 7)
UUUUCUGGUUUCUGUGCCACACUCAGU,
(SEQ ID NO: 9)
UUUUUCUGGUUUCUGUGCCACACUCAG,
(SEQ ID NO: 11)
GUUUUUCUGGUUUCUGUGCCACACUCA,
(SEQ ID NO: 13)
AGUUUUUCUGGUUUCUGUGCCACACUC,
(SEQ ID NO: 15)
AAGUUUUUCUGGUUUCUGUGCCACACU,
(SEQ ID NO: 17)
GCAGGCCAGUACUUGCAGCGCUCCAAA,
(SEQ ID NO: 19)
AAGCAGGGAAGUAACGGCAGAGCUGAC.
(SEQ ID NO: 21)
CAAGCAGGGAAGUAACGGCAGAGCUGA,
(SEQ ID NO: 23)
ACAAGCAGGGAAGUAACGGCAGAGCUG,
(SEQ ID NO: 25)
UACAAGCAGGGAAGUAACGGCAGAGCU
(SEQ ID NO: 27)
UUACAAGCAGGGAAGUAACGGCAGAGC,
(SEQ ID NO: 29)
CUUACAAGCAGGGAAGUAACGGCAGAG,
(SEQ ID NO: 31)
UCUUACAAGCAGGGAAGUAACGGCAGA,
(SEQ ID NO: 33)
UUCUUACAAGCAGGGAAGUAACGGCAG,
(SEQ ID NO: 35)
CACUCAUCUCAGCGUUAGAAAAGCUACC,
(SEQ ID NO: 37)
UCUGGUUUCUGUGCCACACUCAGUUCAC,
(SEQ ID NO: 39)
UACUUGCAGCGCUCCAAAAGUUUUUCUG,
(SEQ ID NO: 41)
UCCCCAUUUUUACAAGCAGGCCAGUACU,
(SEQ ID NO: 43)
GAUGGGGUGAUGGUAGGCACACUCAUCC,
(SEQ ID NO: 45)
UUGGGGAAGGCUUUGCAGGGUGAGAUGG,
(SEQ ID NO: 47)
AAACAUUUUUCAGCAAAUUUACAAUUGG,
(SEQ ID NO: 49)
UAUUUACAAUUUGGGUGAACAAACAAAC,
(SEQ ID NO: 51)
UCUGGUUUAGUACACUUUGCAUCAUAUU,
(SEQ ID NO: 53)
UACUCACAUGAGUGAAGGGACAAUCUGG,
(SEQ ID NO: 55)
UUGGAGACAGUACUGGAAUUCUUCUACU,
(SEQ ID NO: 57)
GUGGUGCUGGUGGUGCAACUGGUUUUGG,
(SEQ ID NO: 59)
ACGGCAGAGCUGACUACUGGAAGGUGGU,
(SEQ ID NO: 61)
CCAUCUUCUUACAAGCAGGGAAGUAACGG,
(SEQ ID NO: 63)
GUUUUGGAUGAUAGAAGGGACAUUCCAU,
(SEQ ID NO: 65)
UACAUUGAGUGUUAAACCUACAAUGUUU,
(SEQ ID NO: 67)
GUAGAAUGUGCAGUCCGGUCUUGUACAU,
(SEQ ID NO: 69)
UGGUGGGACAUUAAUGGUGGGAUGGUAG,
(SEQ ID NO: 71)
UCGAAUCCAUUUCAAGGCAUGUCGUGGU,
or
(SEQ ID NO: 73)
UUAUUCGCUGGUUUGAGGUCGAAUCCAU.

Disclosed herein, are pharmaceutical compositions, comprising a nucleic acid sequence or molecule wherein the nucleic acid comprises or consists of a sequence having at least 90% identity to the sequence set forth in:

(SEQ ID NO: 7)
UUUUCUGGUUUCUGUGCCACACUCAGU,
(SEQ ID NO: 9)
UUUUUCUGGUUUCUGUGCCACACUCAG,
(SEQ ID NO: 11)
GUUUUUCUGGUUUCUGUGCCACACUCA,
(SEQ ID NO: 13)
AGUUUUUCUGGUUUCUGUGCCACACUC,
(SEQ ID NO: 15)
AAGUUUUUCUGGUUUCUGUGCCACACU,
(SEQ ID NO: 17)
GCAGGCCAGUACUUGCAGCGCUCCAAA,
(SEQ ID NO: 19)
AAGCAGGGAAGUAACGGCAGAGCUGAC.
(SEQ ID NO: 21)
CAAGCAGGGAAGUAACGGCAGAGCUGA,
(SEQ ID NO: 23)
ACAAGCAGGGAAGUAACGGCAGAGCUG,
(SEQ ID NO: 25)
UACAAGCAGGGAAGUAACGGCAGAGCU
(SEQ ID NO: 27)
UUACAAGCAGGGAAGUAACGGCAGAGC,
(SEQ ID NO: 29)
CUUACAAGCAGGGAAGUAACGGCAGAG,
(SEQ ID NO: 31)
UCUUACAAGCAGGGAAGUAACGGCAGA,
(SEQ ID NO: 33)
UUCUUACAAGCAGGGAAGUAACGGCAG,
(SEQ ID NO: 35)
CACUCAUCUCAGCGUUAGAAAAGCUACC,
(SEQ ID NO: 37)
UCUGGUUUCUGUGCCACACUCAGUUCAC,
(SEQ ID NO: 39)
UACUUGCAGCGCUCCAAAAGUUUUUCUG,
(SEQ ID NO: 41)
UCCCCAUUUUUACAAGCAGGCCAGUACU,
(SEQ ID NO: 43)
GAUGGGGUGAUGGUAGGCACACUCAUCC,
(SEQ ID NO: 45)
UUGGGGAAGGCUUUGCAGGGUGAGAUGG,
(SEQ ID NO: 47)
AAACAUUUUUCAGCAAAUUUACAAUUGG,
(SEQ ID NO: 49)
UAUUUACAAUUUGGGUGAACAAACAAAC,
(SEQ ID NO: 51)
UCUGGUUUAGUACACUUUGCAUCAUAUU,
(SEQ ID NO: 53)
UACUCACAUGAGUGAAGGGACAAUCUGG,
(SEQ ID NO: 55)
UUGGAGACAGUACUGGAAUUCUUCUACU,
(SEQ ID NO: 57)
GUGGUGCUGGUGGUGCAACUGGUUUUGG,
(SEQ ID NO: 59)
ACGGCAGAGCUGACUACUGGAAGGUGGU,
(SEQ ID NO: 61)
CCAUCUUCUUACAAGCAGGGAAGUAACGG,
(SEQ ID NO: 63)
GUUUUGGAUGAUAGAAGGGACAUUCCAU,
(SEQ ID NO: 65)
UACAUUGAGUGUUAAACCUACAAUGUUU,
(SEQ ID NO: 67)
GUAGAAUGUGCAGUCCGGUCUUGUACAU,
(SEQ ID NO: 69)
UGGUGGGACAUUAAUGGUGGGAUGGUAG,
(SEQ ID NO: 71)
UCGAAUCCAUUUCAAGGCAUGUCGUGGU,
or
(SEQ ID NO: 73)
UUAUUCGCUGGUUUGAGGUCGAAUCCAU.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the nucleic acids, vectors, siRNAs, antisense siRNAs, and sense siRNAs described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions as disclosed herein can be prepared for oral or parenteral administration. Pharmaceutical compositions prepared for parenteral administration include those prepared for intravenous (or intra-arterial), intramuscular, subcutaneous, intrathecal or intraperitoneal administration. Paternal administration can be in the form of a single bolus dose, or may be, for example, by a continuous pump. In some aspects, the compositions can be prepared for parenteral administration that includes dissolving or suspending the nucleic acids, polynucleic sequences, vectors or siRNA molecules in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

In some aspects, the compositions disclosed herein are formulated for oral, intramuscular, intravenous, subcutaneous, intrathecal or intraperitoneal administration.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be siRNA molecules, nucleic acids or vectors described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

In some aspects, the vectors, siRNAs and nucleic acid sequences as disclosed herein can be delivered to a cell of the subject. In some aspects, such action can be achieved, for example, by using polymeric, biodegradable microparticle or microcapsule delivery vehicle, sized to optimize phagocytosis by phagocytic cells (e.g., macrophages).

In some aspects, the formulations include any that are suitable for the delivery of a virus (e.g., adeno-associated virus) and cells. In some aspects, the route of administration includes but is not limited to direct injection into the brain. Such administration can be done without surgery, or with surgery.

### Kits

Disclosed herein are kits that comprise any combination of the compositions (e.g., any of siRNAs) described above and suitable instructions (e.g., written and/or provided as audio-, visual-, or audiovisual material). Disclosed herein are kits that comprise any combination of the pharmaceutical compositions described above and suitable instructions (e.g., written and/or provided as audio-, visual-, or audiovisual material). In some aspects, the kit comprises a predetermined amount of a composition or pharmaceutical composition comprising any of the siRNA molecules disclosed herein. The kit can further comprise one or more of the following: instructions, sterile fluid, syringes, a sterile container, delivery devices, and buffers or other control reagents.

### EXAMPLES

### Example 1: Targeted Nucleic Acid Sequences for Silencing MSUT2/ZC3H14

HEK293 cells were cultured under standard tissue culture conditions (DMEM, 10% defined fetal bovine serum, Penicillin (1000 IU/mL) Streptomycin (1000 mg/mL) (Wheeler et al., Science Translational Medicine, 2019 Dec. 18; 11(253)). RNA interference transfections were conducted following the manufacturer's protocol (RNAiMAX, Invitrogen). Cell pellet lysates were prepared for immunodetection (Wheeler et al., Science Translational Medicine, 2019 Dec. 18; 11(253)). Lysates were diluted in 0.1× sample buffer (1:25; Protein Simple) and analyzed on a Peggy Sue (Protein Simple) following manufacturer's protocols using 12-230 kDa capillaries. MSUT2 was detected with the Rbt9857 antibody (Wheeler, et al 2019 (STM)) diluted at 1:10 in Antibody Diluent 2 (Protein Simple) and actin was detected with A4700 (SigmaAldrich) diluted at 1:200. Goat anti-rabbit secondary antibody (GE Lifescience) was diluted to 1:100 in Antibody Diluent 2. MSUT2 knockdown was analyzed by peak height and peak area normalized to actin.

To measure the effectiveness of siRNA treatments, synthetic siRNAs were introduced into HEK293 cells using lipofectamine RNAimax reagent (Thermo) according to the manufacturer's instructions. Three days post transfection siRNA treated cells were harvested and analyzed for MSUT2 protein levels using a ProteinSimple capillary immunoanalzyer. MSUT2 protein levels were compared to MSUT2 siRNA and mock treated cells and expressed as a percentage of endogenous MSUT2 levels. The results are shown in Table 3.

TABLE 3

| | | | | | | % KD-MSUT2 |
|---|---|---|---|---|---|---|
| | | | | | | relative |
| | | | SEQ | | SEQ | to actin |
| | | | ID | Anti- | ID | by Sally |
| Sample | Name | Sense | NO: | Sense | NO: | Assay |
| 1 | Standard | ATGATGC | 77 | TCTGGTTT | 78 | 75.83280863 |
| | MSUT2 | AAAGTGA | | AGTACACT | | |
| | RNAi | CTAAACC | | TTGCATCA | | |
| | | AG | | TAT | | |
| 3 | MnH MS | UGAGUGU | 6 | UUUUCUGG | 7 | 77.00088007 |
| | UU2si4 | GGCACAG | | UUUCUGUG | | |
| | | AAACCAG | | CCACACUC | | |
| | | AAAA | | AGU | | |
| 4 | MnH MS | GAGUGUG | 8 | UUUUUCUG | 9 | 79.82342853 |
| | UU2si5 | GCACAGA | | GUUUCUGU | | |
| | | AACCAGA | | GCCACACU | | |
| | | AAAA | | CAG | | |
| 5 | MnH MS | AGUGUGG | 10 | GUUUUUCU | 11 | 77.56392345 |
| | UU2si6 | CACAGAA | | GGUUUCUG | | |
| | | ACCAGAA | | UGCCACAC | | |
| | | AAAC | | UCA | | |
| 6 | MnH MS | GUGUGGC | 12 | AGUUUUUC | 13 | 82.96989508 |
| | UU2si7 | ACAGAAA | | UGGUUUCU | | |
| | | CCAGAAA | | GUGCCACA | | |
| | | AACU | | CUC | | |
| 7 | MnH MS | UGUGGCA | 14 | AAGUUUUU | 15 | 77.52231763 |
| | UU2si8 | CAGAAAC | | CUGGUUUC | | |
| | | CAGAAAA | | UGUGCCAC | | |
| | | ACUU | | ACU | | |
| 8 | MnH MS | UGGAGCG | 16 | GCAGGCCA | 17 | 13.8614351 |
| | UU2si9 | CUGCAAG | | GUACUUGC | | |
| | | UACUGGC | | AGCGCUCC | | |
| | | CUGC | | AAA | | |
| 9 | MnH MS | CAGCUCU | 18 | AAGCAGGG | 19 | 73.02265734 |
| | UU2si10 | GCCGUUA | | AAGUAACG | | |
| | | CUUCCCU | | GCAGAGCU | | |
| | | GCUU | | GAC | | |
| 10 | MnH MS | AGCUCUG | 20 | CAAGCAGG | 21 | 79.91434341 |
| | UU2si11 | CCGUUAC | | GAAGUAAC | | |
| | | UUCCCUG | | GGCAGAGC | | |
| | | CUUG | | UGA | | |
| 11 | MnH MS | GCUCUGC | 22 | ACAAGCAG | 23 | 79.26631989 |
| | UU2si12 | CGUUACU | | GGAAGUAA | | |
| | | UCCCUGC | | CGGCAGAG | | |
| | | UUGU | | CUG | | |
| 12 | MnH MS | CUCUGCC | 24 | UACAAGCA | 25 | 67.73347845 |
| | UU2si13 | GUUACUU | | GGGAAGUA | | |
| | | CCCUGCU | | ACGGCAGA | | |
| | | UGUA | | GCU | | |
| 13 | MnH MS | UCUGCCG | 26 | UUACAAGC | 27 | 75.19836445 |
| | UU2si14 | UUACUUC | | AGGGAAGU | | |
| | | CCUGCUU | | AACGGCAG | | |
| | | GUAA | | AGC | | |
| 14 | MnH MS | CUGCCGU | 28 | CUUACAAG | 29 | 78.56434308 |
| | UU2si15 | UACUUCC | | CAGGGAAG | | |
| | | CUGCUUG | | UAACGGCA | | |
| | | UAAG | | GAG | | |
| 15 | MnH MS | UGCCGUU | 30 | UCUUACAA | 31 | 67.74655341 |
| | UU2si16 | ACUUCCC | | GCAGGGAA | | |
| | | UGCUUGU | | GUAACGGC | | |
| | | AAGA | | AGA | | |
| 16 | MnH MS | GCCGUUA | 32 | UUCUUACA | 33 | 56.89172238 |
| | UU2si17 | CUUCCCU | | AGCAGGGA | | |
| | | GCUUGUA | | AGUAACGG | | |
| | | AGAA | | CAG | | |
| 17 | hMSsiwalk | GGUAGCU | 34 | CACUCAUC | 35 | 55.32188634 |
| | 28 | UUUCUAA | | UCAGCGUU | | |
| | | CGCUGAG | | AGAAAAGC | | |
| | | AUGAGUG | | UACC | | |
| 18 | hMSsiwalk | GUGAACU | 36 | UCUGGUUU | 37 | 80.62947589 |
| | 53 | GAGUGUG | | CUGUGCCA | | |
| | | GCACAGA | | CACUCAGU | | |
| | | AACCAGA | | UCAC | | |
| 19 | hMSsiwalk | CAGAAAA | 38 | UACUUGCA | 39 | 52.02312009 |
| | 77 | ACUUUUG | | GCGCUCCA | | |
| | | GAGCGCU | | AAAGUUUU | | |
| | | GCAAGUA | | UCUG | | |
| 20 | hMSsiwalk | AGUACUG | 40 | UCCCCAUU | 41 | 70.07765806 |
| | 101 | GCCUGCU | | UUUACAAG | | |
| | | UGUAAAA | | CAGGCCAG | | |
| | | AUGGGGA | | UACU | | |
| 21 | hMSsiwalk | GGAUGAG | 42 | GAUGGGGU | 43 | 23.16656271 |
| | 126 | UGUGCCU | | GAUGGUAG | | |
| | | ACCAUCA | | GCACACUC | | |
| | | CCCCAUC | | AUCC | | |
| 22 | hMSsiwalk | CCAUCUC | 44 | UUGGGGAA | 45 | 82.6631096 |
| | 149 | ACCCUGC | | GGCUUUGC | | |
| | | AAAGCCU | | AGGGUGAG | | |
| | | UCCCCAA | | AUGG | | |
| 23 | hMSsiwalk | CCAAUUG | 46 | AAACAUUU | 47 | 66.00892982 |
| | 173 | UAAAUUU | | UUCAGCAA | | |
| | | GCUGAAA | | AUUUACAA | | |
| | | AAUGUUU | | UUGG | | |
| 24 | hMSsiwalk | GUUUGUU | 48 | UAUUUACA | 49 | 56.22027712 |
| | 197 | UGUUCAC | | AUUUGGGU | | |
| | | CCAAAUU | | GAACAAAC | | |
| | | GUAAAUA | | AAAC | | |
| 25 | hMSsiwalk | AAUAUGA | 50 | UCUGGUUU | 51 | 71.61900312 |
| | 221 | UGCAAAG | | AGUACACU | | |
| | | UGUACUA | | UUGCAUCA | | |
| | | AACCAGA | | UAUU | | |
| 26 | hMSsiwalk | CCAGAUU | 52 | UACUCACA | 53 | 74.78356031 |
| | 244 | GUCCCUU | | UGAGUGAA | | |
| | | CACUCAU | | GGGACAAU | | |
| | | GUGAGUA | | CUGG | | |
| 27 | hMSsiwalk | AGUAGAA | 54 | UUGGAGAC | 55 | 82.55942726 |
| | 268 | GAAUUCC | | AGUACUGG | | |
| | | AGUACUG | | AAUUCUUC | | |
| | | UCUCCAA | | UACU | | |
| 28 | hMSsiwalk | CCAAAAC | 56 | GUGGUGCU | 57 | 69.89754374 |
| | 292 | CAGUUGC | | GGUGGUGC | | |
| | | ACCACCA | | AACUGGUU | | |
| | | GCACCAC | | UUGG | | |
| 29 | hMSsiwalk | ACCACCU | 58 | ACGGCAGA | 59 | 74.63956705 |
| | 315 | UCCAGUA | | GCUGACUA | | |
| | | GUCAGCU | | CUGGAAGG | | |
| | | CUGCCGU | | UGGU | | |
| 30 | hMSsiwalk | CCGUUAC | 60 | CCAUCUUC | 61 | 76.18952012 |
| | 340 | UUCCCUG | | UUACAAGC | | |
| | | CUUGUAA | | AGGGAAGU | | |
| | | GAAGAUG | | AACGG | | |
| | | G | | | | |
| 31 | hMSsiwalk | AUGGAAU | 62 | GUUUUGGA | 63 | 78.9692047 |
| | 364 | GUCCCUU | | UGAUAGAA | | |
| | | CUAUCAU | | GGGACAUU | | |
| | | CCAAAAC | | CCAU | | |
| 32 | hMSsiwalk | AAACAUU | 64 | UACAUUGA | 65 | 81.47011055 |
| | 388 | GUAGGUU | | GUGUUAAA | | |
| | | UAACACU | | CCUACAAU | | |
| | | CAAUGUA | | GUUU | | |
| 33 | hMSsiwalk | AUGUACA | 66 | GUAGAAUG | 67 | 53.31365239 |
| | 411 | AGACCGG | | UGCAGUCC | | |
| | | ACUGCAC | | GGUCUUGU | | |
| | | AUUCUAC | | ACAU | | |
| 34 | hMSsiwalk | CUACCAU | 68 | UGGUGGGA | 69 | 64.99244605 |
| | 408 | CCCACCA | | CAUUAAUG | | |
| | | UUAAUGU | | GUGGGAUG | | |
| | | CCCACCA | | GUAG | | |
| 35 | hMSsiwalk | ACCACGA | 70 | UCGAAUCC | 71 | 68.24885441 |
| | 432 | CAUGCCU | | AUUUCAAG | | |
| | | UGAAAUG | | GCAUGUCG | | |
| | | GAUUCGA | | UGGU | | |
| 36 | hMSsiwalk | AUGGAUU | 72 | UUAUUCGC | 73 | 68.45608284 |
| | 450 | CGACCUC | | UGGUUUGA | | |
| | | AAACCAG | | GGUCGAAU | | |
| | | CGAAUAA | | CCAU | | |

What is claimed is:

1. A composition comprising a nucleic acid sequence or molecule wherein the nucleic acid comprises or consists of a sequence having at least 90% identity to the sequence set forth in:
(SEQ ID NO: 7)
UUUUCUGGUUUCUGUGCCACACUCAGU,
(SEQ ID NO: 9)
UUUUUCUGGUUUCUGUGCCACACUCAG,
(SEQ ID NO: 11)
GUUUUUCUGGUUUCUGUGCCACACUCA,
(SEQ ID NO: 13)
AGUUUUUCUGGUUUCUGUGCCACACUC,
(SEQ ID NO: 15)
AAGUUUUUCUGGUUUCUGUGCCACACU,
(SEQ ID NO: 17)
GCAGGCCAGUACUUGCAGCGCUCCAAA,
(SEQ ID NO: 19)
AAGCAGGGAAGUAACGGCAGAGCUGAC.
(SEQ ID NO: 21)
CAAGCAGGGAAGUAACGGCAGAGCUGA,
(SEQ ID NO: 23)
ACAAGCAGGGAAGUAACGGCAGAGCUG,
(SEQ ID NO: 25)
UACAAGCAGGGAAGUAACGGCAGAGCU
(SEQ ID NO: 27)
UUACAAGCAGGGAAGUAACGGCAGAGC,
(SEQ ID NO: 29)
CUUACAAGCAGGGAAGUAACGGCAGAG,
(SEQ ID NO: 31)
UCUUACAAGCAGGGAAGUAACGGCAGA,
(SEQ ID NO: 33)
UUCUUACAAGCAGGGAAGUAACGGCAG,
(SEQ ID NO: 35)
CACUCAUCUCAGCGUUAGAAAAGCUACC,
(SEQ ID NO: 37)
UCUGGUUUCUGUGCCACACUCAGUUCAC,
(SEQ ID NO: 39)
UACUUGCAGCGCUCCAAAAGUUUUUCUG,
(SEQ ID NO: 41)
UCCCCAUUUUUACAAGCAGGCCAGUACU,
(SEQ ID NO: 43)
GAUGGGGUGAUGGUAGGCACACUCAUCC,
(SEQ ID NO: 45)
UUGGGGAAGGCUUUGCAGGGUGAGAUGG,
(SEQ ID NO: 47)
AAACAUUUUUCAGCAAAUUUACAAUUGG,
(SEQ ID NO: 49)
UAUUUACAAUUUGGGUGAACAAACAAAC,
(SEQ ID NO: 51)
UCUGGUUUAGUACACUUUGCAUCAUAUU,
(SEQ ID NO: 53)
UACUCACAUGAGUGAAGGGACAAUCUGG,
(SEQ ID NO: 55)
UUGGAGACAGUACUGGAAUUCUUCUACU,
(SEQ ID NO: 57)
GUGGUGCUGGUGGUGCAACUGGUUUUGG,
(SEQ ID NO: 59)
ACGGCAGAGCUGACUACUGGAAGGUGGU,
(SEQ ID NO: 61)
CCAUCUUCUUACAAGCAGGGAAGUAACGG,
(SEQ ID NO: 63)
GUUUUGGAUGAUAGAAGGGACAUUCCAU,
(SEQ ID NO: 65)
UACAUUGAGUGUUAAACCUACAAUGUUU,
(SEQ ID NO: 67)
GUAGAAUGUGCAGUCCGGUCUUGUACAU,
(SEQ ID NO: 69)
UGGUGGGACAUUAAUGGUGGGAUGGUAG,
(SEQ ID NO: 71)
UCGAAUCCAUUUCAAGGCAUGUCGUGGU,
or
(SEQ ID NO: 73)
UUAUUCGCUGGUUUGAGGUCGAAUCCAU.

2. A siRNA molecule wherein the siRNA molecule specifically targets at least one sequence selected from the group consisting of SEQ ID NO: 1 to SEQ ID NO: 5, SEQ ID NO: 77 and SEQ ID NO: 78 and reduces expression of mammalian suppressor of tauopathy 2 (MSUT2) gene in a cell, wherein the siRNA molecule comprises a 25- to 28-nucleotide blunt-ended double-stranded structure, wherein the siRNA molecule comprises at least one sequence having at least 90% sequence identity selected from the group consisting of SEQ ID NO: 6 to SEQ ID NO: 73.

3. The siRNA molecule of claim 2, wherein at least one nucleotide of the siRNA molecule comprises a chemical modification.

4. The siRNA molecule of claim 3, wherein the chemical modification is on the sense strand, the antisense strand or on both strands of the siRNA molecule.

5. The siRNA molecule of claim 2, wherein the siRNA molecule comprises at least one sequence selected from the group consisting of SEQ ID NO: 6-SEQ ID NO: 73.

6. The siRNA molecule of claim 2, wherein the siRNA molecule comprises or consists of a sense strand which comprises or consists of at least one sequence selected from the group of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64 SEQ ID NO: 66, SEQ ID NO: 68, SEQ ID NO: 70, and SEQ ID NO: 72; and an antisense strand which is complementary to the sense strand which is selected from the group of SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65 SEQ ID NO: 67, SEQ ID NO: 69, SEQ ID NO: 71, and SEQ ID NO: 73.

7. A pharmaceutical composition, wherein the composition comprises at least one siRNA molecule according to any of the preceding claims.

8. The composition of claim 1, further comprising a pharmaceutically acceptable carrier.

9. The composition of claim 8, wherein the pharmaceutically acceptable carrier comprises a lipid-based or polymer-based colloid.

10. The composition of claim 9, wherein the colloid is a liposome, a hydrogel, a microparticle, a nanoparticle, or a block copolymer micelle.

11. The siRNA molecule of claims 2 to 6, further comprising a pharmaceutically acceptable carrier.

12. The siRNA molecule of claim 11, wherein the pharmaceutically acceptable carrier comprises a lipid-based or polymer-based colloid.

13. The siRNA molecule of claim 12, wherein the siRNA molecule is formulated for intravenous, subcutaneous or intrathecal administration.

14. A method of treating Alzheimer's disease or dementia, the method comprising:
administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, and wherein the therapeutically effective amount reduces accumulation of phosphorylated and aggregated human tau.

15. A method of inhibiting expression of a MSUT2 polynucleotide in a subject, the method comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

16. A method of reducing phosphorylated and aggregated human tau protein in a subject, the method comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

17. A method of suppressing expression of a MSUT2 polynucleotide in a subject, the method comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

18. A method of potentiating a neuroinflammatory response to a pathological tau protein in a subject, the method comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

19. A method of decreasing astrocytosis or microgliosis in a subject, the method comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

20. A method of reducing neuroinflammation in a subject, the method comprising administering to a subject with Alzheimer's disease or dementia a therapeutically effective amount of a small interfering RNA (siRNA) molecule or a composition comprising the siRNA molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73.

21. The method of any of claims 14-20, wherein the subject is identified as being in need of treatment before the administration step.

22. The method of any of claims 14-21, wherein the subject is a human.

23. The method of any of claims 14-22, further comprising administering a cholinesterase inhibitor to the subject.

24. The method of claim 23, wherein the cholinesterase inhibitor is galantamine, rivastigmine or donepezil.

25. The method of any of claims 14-24, wherein the subject has Alzheimer's disease.

26. The method of claim 25, wherein the subject has mild-moderate Alzheimer's disease.

27. The method of claim 25, wherein the subject has moderate-severe Alzheimer's disease.

28. The method of any of claims 14-24, wherein the subject has dementia.

29. The method of any of claims 14-28, wherein the composition further comprises a pharmaceutically acceptable carrier.

30. The method of claim 29, wherein the pharmaceutically acceptable carrier comprises a lipid-based or polymer-based colloid.

31. The method of any of claims 14-30, wherein the siRNA molecule is formulated for intravenous, subcutaneous or intrathecal administration.

32. The method of any of claims 14-31, wherein the therapeutically effective amount of the siRNA molecule or a composition comprising the siRNA molecule is administered orally, intramuscularly, intraperitoneally, intravenously, subcutaneously or intrathecally.

33. A method of inhibiting expression of a MSUT2 polynucleotide, the method comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

34. A method of suppressing expression of a MSUT2 polynucleotide, the method comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

35. A method of potentiating a neuroinflammatory response to a pathological tau protein, the method comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

36. A method of decreasing astrocytosis or microgliosis, the method comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

37. A method of reducing neuroinflammation, the method comprising contacting a cell with a small interfering RNA (siRNA) molecule that specifically targets at least one sequence selected from the group consisting of SEQ ID NO: 1 to SEQ ID NO: 5, SEQ ID NO: 77 and SEQ ID NO: 78, wherein the siRNA molecule comprises a 25- to 28-nucleotide blunt-ended double-stranded structure, wherein the siRNA molecule comprises at least one sequence selected from the group consisting of SEQ ID NO: 6 to SEQ ID NO: 73, wherein the siRNA molecule reduces accumulation of phosphorylated and aggregated tau.

38. The method of any of claim 15, 17, 33, or 34, wherein the expression of the MSUT2 polynucleotide is inhibited or suppressed by the siRNA molecule is by inhibiting the binding of poly(A) RNA to the MSUT2 polynucleotide.

39. The method of any of claim 33, 34, 35, 36, or 37, wherein the cell is a mammalian cell.

40. The method of claim 39, wherein the mammalian cell is a brain cell.

41. The method of any claims 33-35, wherein at least one nucleotide of the siRNA molecule comprises a chemical modification.

42. The method of claim 41, wherein the chemical modification is on the sense strand, the antisense strand or on both.

43. The method of any claims 33-38, wherein the siRNA molecule comprises at least one sequence is selected from the group consisting of SEQ ID NO: 6-SEQ ID NO: 73.

44. The method of any of the preceding claims, wherein the siRNA molecule comprises or consists of a sense strand which comprises or consists of at least one sequence selected from the group of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64 SEQ ID NO: 66, SEQ ID NO: 68, SEQ ID NO: 70, and SEQ ID NO: 72; and an antisense strand which is complementary to the sense strand which is selected from the group of SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65 SEQ ID NO: 67, SEQ ID NO: 69, SEQ ID NO: 71, and SEQ ID NO: 73.
